(19) United States
(12) Patent Application Publication
(10) Pub. No.: US 2024/0245345 A1
(43) Pub. Date: Jul. 25, 2024

(54) NON-INVASIVE DETECTION AND DIFFERENTIATION OF SEPSIS

(71) Applicant: Flashback Technologies, Inc., Vienna, VA (US)

(72) Inventors: John Jedziniak, Greenwood Village, CO (US); Abhishek Jaiantilal, Highlands Ranch, CO (US); Mike Halperin, Seattle, WA (US)

(21) Appl. No.: 18/560,189

(22) PCT Filed: May 10, 2022
(86) PCT No.: PCT/US22/28493

§ 371 Date: Nov. 10, 2023

Related U.S. Application Data
(60) Provisional application No. 63/186,327, filed on May 10, 2021.

(51) Int. Cl. A61B 5/00; A61B 5/02; A61B 5/0205; G16H 20/10; G16H 40/63; G16H 50/20; G16H 50/30; G16H 50/50

(52) U.S. Cl. CPC A61B 5/412; A61B 5/02028; A61B 5/02042; A61B 5/02055; A61B 5/4839; A61B 5/7246; A61B 5/7275; A61B 5/742; G16H 20/10; G16H 40/63; G16H 50/20; G16H 50/30; G16H 50/50

(57) ABSTRACT

Novel tools and techniques are provided for detecting and differentiating sepsis are provided A system includes one or more sensors configured to obtain non-invasive physiological data from a patient, and estimate a compensatory reserve index based on the physiological data. A computer system may determine, based on the compensatory reserve index, whether the patient is septic by applying a sepsis model configured to relate hemodynamic parameters over time to a determination of sepsis. Hemodynamic parameters may include a compensatory reserve index (CRI).

### CROSS-REFERENCE TO RELATED APPLICATIONS

This application claims priority to U.S. Provisional Patent Application Ser. No. 63/186,327, filed May 10, 2021 by John Jedziniak et al. (attorney docket no. 0463.21PR), entitled “NON-INVASIVE DETECTION AND DIFFERENTIATION OF SEPSIS,” the disclosures of which are incorporated herein by reference in their entirety for all purposes.

This application may be related to U.S. patent application Ser. No. 15/620,701, filed Jun. 12, 2017 by Mulligan et al. and entitled “Rapid Detection of Bleeding Following Injury” (attorney docket no. 0463.17, referred to herein as the “'701 Application”), which claims priority to provisional U.S. Patent Application No. 62/349,516, filed Jun. 13, 2016 by Mulligan et al. and entitled “Rapid Detection of Bleeding Following Injury” (attorney docket no. 0463.17PR, referred to herein as the “'516 Application”), each of which is incorporated herein by reference in its entirety. This application may also be related to U.S. patent application Ser. No. 15/261,661, filed Sep. 9, 2016 by Mulligan et al. and entitled “Estimating Physiological States Based on Changes in CRI” (attorney docket no. 0463.16, referred to herein as the “'661 Application”), which claims priority to the '516 Application and to provisional U.S. Patent Application No. 62/216,187, filed Sep. 9, 2015 by Mulligan et al. and entitled “Estimating Physiological States Based on Changes in CRI” (attorney docket no. 0463.16PR, referred to herein as the “'187 Application”), each of which is incorporated herein by reference in its entirety.

This application may also be related to U.S. patent application Ser. No. 14/885,891, filed Oct. 16, 2015 by Mulligan et al. and entitled “Assessing Effectiveness of CPR” (attorney docket no. 0463.15, referred to herein as the “'891 Application”) and U.S. patent application Ser. No. 14/885,888, filed Oct. 16, 2015 by Mulligan et al. and entitled “Rapid Detection of Bleeding Before, During, and After Fluid Resuscitation” (attorney docket no. 0463.14, referred to herein as the “'888 Application”), each of which claims priority to provisional U.S. Patent Application No. 62/064,816, filed Oct. 16, 2014 by Mulligan et al. and titled “Assessing the Effectiveness of CPR” (attorney docket no. 0463.15PR, referred to herein as the “'816 Application”) and provisional U.S. Patent Application No. 62/064,809 filed Oct. 16, 2014 by Mulligan et al. and titled “Rapid Detection of Bleeding During Fluid Resuscitation” (attorney docket no. 0463.14PR, referred to herein as the “'809 Application”), each of which is incorporated herein by reference in its entirety.

This application may also be related to U.S. patent application Ser. No. 14/542,426, filed Nov. 14, 2014 by Mulligan et al. and titled, “Noninvasive Hydration Monitoring” (attorney docket no. 0463.12, referred to herein as the “'426 Application”) and U.S. patent application Ser. No. 14/542,423, filed Nov. 14, 2014 by Mulligan et al. and titled, “Noninvasive Monitoring for Fluid Resuscitation” (attorney docket no. 0463.11, referred to herein as the “'423 Application”), each of which claims priority to provisional U.S. Patent Application No. 61/905,727, filed Nov. 18, 2013 by Mulligan et al. and titled “Noninvasive Hydration Monitoring” (attorney docket no. 0463.12PR, referred to herein as the “'727 Application”) and provisional U.S. Patent Application No. 61/904,436, filed Nov. 14, 2013 by Mulligan et al. and titled “Noninvasive Monitoring for Fluid Resuscitation” (attorney docket no. 0463.11PR, referred to herein as the “'436 Application”), each of which is incorporated herein by reference in its entirety.

This application may also be related to U.S. patent application Ser. No. 14/535,171, filed Nov. 6, 2014 by Mulligan et al. and titled “Noninvasive Predictive and/or Estimative Blood Pressure Monitoring” (attorney docket no. 0463.10, referred to herein as the “'171 Application”), which claims priority to the '727 Application, the '436 Application, and provisional U.S. Patent Application No. 61/900,980, filed Nov. 6, 2013 by Mulligan et al. and titled “Noninvasive Predictive and/or Estimative Blood Pressure Monitoring” (attorney docket no. 0463.10PR), each of which is incorporated herein by reference in its entirety.

This application may also be related to U.S. patent application Ser. No. 13/554,483, filed Jul. 20, 2012 by Grudic et al. and titled, “Hemodynamic Reserve Monitor and Hemodialysis Control” (attorney docket no. 0463.05, referred to herein as the “'483 Application”; now issued U.S. Pat. No. 9,757,041), which claims priority to provisional U.S. Patent Application No. 61/510,792, filed Jul. 22, 2011 by Grudic et al. and entitled “Cardiovascular Reserve Monitor” (attorney docket no. 0463.05PR, referred to herein as the “'792 Application”) and provisional U.S. Patent Application No. 61/614,426, filed Mar. 22, 2012 by Grudic et al. and entitled “Hemodynamic Reserve Monitor and Hemodialysis Control” (attorney docket no. 0463.07PR, referred to herein as the “'426 Application”), each of which is hereby incorporated by reference in its entirety.

This application may also be related to U.S. patent application Ser. No. 13/041,006, filed Mar. 4, 2011 by Grudic et al. and entitled “Active Physical Perturbations to Enhance Intelligent Medical Monitoring” (attorney docket no. 0463.04, referred to herein as the “'006 Application”), which claims priority to provisional U.S. Patent Application No. 61/310,583, filed Mar. 4, 2010 by Grudic et al. and titled “Active Physical Perturbations to Enhance Intelligent Medical Monitoring” (attorney docket no. 0463.04PR, referred to herein as the “'583 Application”), each of which is hereby incorporated by reference in its entirety.

This application may also be related to U.S. patent application Ser. No. 13/028,140, filed Feb. 15, 2011 by Grudic et al. and entitled “Statistical, Noninvasive Measurement of Intracranial Pressure” (attorney docket no. 0463.03, referred to herein as the “'140 Application”; now issued U.S. Pat. No. 8,512,260), which claims priority to provisional U.S. Patent Application No. 61/305,110, filed Feb. 16, 2010, by Moulton et al. and titled “Statistical, Noninvasive Method for Predicting Intracranial Pressure” (attorney docket no. 0463.03PR, referred to herein as the “'110 Application”), each of which is hereby incorporated by reference in its entirety.

This application may also be related to International Application No. PCT/US2009/062119, filed Oct. 26, 2009 by Grudic et al. and entitled “Long Term Active Learning from Large Continually Changing Data Sets” (attorney docket no. 0463.01/PCT, referred to herein as the “'119 Application”), which claims priority to provisional U.S. Patent Application No. 61/252,978, filed Oct. 19, 2009 by Grudic et al. and titled “Long Term Active Learning from Large Continually Changing Data Sets,” provisional U.S. Patent Application No. 61/166,499, filed Apr. 3, 2009 by Moulton and titled “Advances in Pre-Hospital Care,” provisional U.S. Patent Application No. 61/166,486, filed Apr. 3, 2009 by Grudic et al. and titled “Statistical Methods for Predicting Patient Specific Blood Loss Volume Causing Hemodynamic Decompensation,” provisional U.S. Patent Application No. 61/166,472, filed Apr. 3, 2009 by Grudic et al. and titled “Long Term Active Learning from Large Continually Changing Data Sets,” and provisional U.S. Patent Application No. 61/109,490, filed Oct. 29, 2008 by Moulton et al. and titled “Method for Determining Physiological State or Condition,” each of which is hereby incorporated by reference in its entirety.

The respective disclosures of these applications/patents (which this document refers to collectively as the “Related Applications”) are incorporated herein by reference in their entirety for all purposes.

### COPYRIGHT STATEMENT

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

### FIELD

The present disclosure relates, in general, to methods, systems, and apparatuses for implementing physiological monitoring, and, more particularly, to methods, systems, and apparatuses for detecting and differentiating sepsis and septic shock in a patient.

### BACKGROUND

Sepsis is a life-threatening condition caused by a dysregulated host immune response to infection. Sepsis and septic shock (sepsis which has progressed to a shock state) are one of the most pressing diseases facing modern medicine. In 2017, 48.9 million cases of sepsis and 11 million sepsis-related deaths were recorded worldwide. The COVID pandemic has further highlighted the urgent need for paradigm shifting technology that allows medical personnel to quickly recognize and initiate early treatment of sepsis.

Therefore, methods, systems, and apparatuses for quickly detecting sepsis and differentiating sepsis from other conditions are provided.

### BRIEF DESCRIPTION OF THE DRAWINGS

A further understanding of the nature and advantages of particular embodiments may be realized by reference to the remaining portions of the specification and the drawings, in which like reference numerals are used to refer to similar components. In some instances, a sub-label is associated with a reference numeral to denote one of multiple similar components. When reference is made to a reference numeral without specification to an existing sub-label, it is intended to refer to all such multiple similar components.

FIG. 1 is a schematic diagram illustrating a system for detecting and differentiating sepsis, in accordance with various embodiments;

FIG. 2 is a schematic diagram illustrating a system for estimating compensatory reserve, which can be used for implement sepsis detection and differentiation, in accordance with various embodiments;

FIG. 3 is a flow diagram illustrating a method of estimating a patient's compensatory reserve, in accordance with various embodiments;

FIG. 4 is a flow diagram illustrating a method of determining whether a patient is septic and differentiating sepsis from other conditions, in accordance with various embodiments; and

FIG. 5 is a block diagram illustrating an exemplary computer or system hardware architecture, in accordance with various embodiments.

### DETAILED DESCRIPTION OF CERTAIN EMBODIMENTS

Various embodiments provide tools and techniques for detecting and differentiating sepsis in a patient using non-invasive monitoring techniques.

In an aspect, a method for detecting sepsis in a patient is provided. The method includes obtaining, with one or more sensors disposed in a sensor device, physiological data of a patient continuously over a first time period, the physiological data comprising non-invasively obtained waveforms of physiological data, and determining, via a computer system, based on the physiological data, a hemodynamic parameter of the patient over the first time period, wherein the hemodynamic parameter is a patient-specific indication of the patient's proximity to hemodynamic decompensation at a given time, wherein the hemodynamic parameter is a numerical value indicating a relationship between an intravascular volume loss of a patient at the given time and an intravascular volume loss at hemodynamic decompensation of the patient. Determining the hemodynamic parameter of the patient may further include applying a hemodynamic model to the physiological data, the hemodynamic model relating the physiological data to the hemodynamic parameter, wherein the hemodynamic model comprises a plurality waveforms of reference data, comparing one or more waveforms of the physiological data of the patient to the each of the plurality of waveforms of reference data, each of the plurality of waveforms of reference data corresponding to a respective value of the hemodynamic parameter, and determining the hemodynamic parameter of the patient based on the comparison to each of the plurality of waveforms of reference data. The method further includes determining, via the computer system, based on the hemodynamic parameter of the patient over the first time period, whether the patient is septic. Determining whether the patient is septic further includes applying a sepsis model to the hemodynamic parameter of the patient over the first time period, wherein the hemodynamic parameter over the first time period is a waveform of the hemodynamic parameter of the patient, the sepsis model relating waveforms of the hemodynamic parameter to a sepsis value representing whether the patient is septic, wherein sepsis model comprises a plurality of reference waveforms of the hemodynamic parameter. The method continues by comparing the waveform of the hemodynamic parameter over the first time period to each of the plurality of reference waveforms of the hemodynamic parameter, each of the plurality of reference waveforms of the hemodynamic parameter corresponding to a respective sepsis value, and determining whether the patient is septic based on the sepsis value of the patient. The method further includes displaying, on a display screen of a user device, at least one of the hemodynamic parameter of the patient and a determination of whether the patient is septic.

In another aspect, an apparatus for detecting sepsis in a patient is provided. The apparatus includes a processor, and a non-transitory computer readable medium in communication with the processor, the non-transitory computer readable medium having encoded thereon a set of instructions executable by the processor to perform various functions. The set of instructions may include instructions that, when executed by the processor, cause the processor to obtain, with one or more sensors disposed in a sensor device, physiological data of a user continuously over a first time period, the physiological data comprising non-invasively obtained waveforms of physiological data, and determine based on the physiological data, a hemodynamic parameter of the patient over the first time period, wherein the hemodynamic parameter is a patient-specific indication of the patient's proximity to hemodynamic decompensation at a given time, wherein the hemodynamic parameter is a numerical value indicating a relationship between an intravascular volume loss of a patient at the given time and an intravascular volume loss at hemodynamic decompensation of the patient. Determining the hemodynamic parameter of the patient may further include applying a hemodynamic model to the physiological data, the hemodynamic model relating the physiological data to the hemodynamic parameter, wherein the hemodynamic model comprises a plurality waveforms of reference data, comparing one or more waveforms of the physiological data of the patient to the each of the plurality of waveforms of reference data, each of the plurality of waveforms of reference data corresponding to a respective value of the hemodynamic parameter, and determining the hemodynamic parameter of the patient based on the comparison to each of the plurality of waveforms of reference data. The set of instructions may further include instructions executable by the processor to determine based on the hemodynamic parameter of the patient over the first time period, whether the patient is septic. Determining whether the patient is septic further includes applying a sepsis model to the hemodynamic parameter of the patient over the first time period, wherein the hemodynamic parameter over the first time period is a waveform of the hemodynamic parameter of the patient, the sepsis model relating waveforms of the hemodynamic parameter to a sepsis value representing whether the patient is septic, wherein sepsis model comprises a plurality of reference waveforms of the hemodynamic parameter, comparing the waveform of the hemodynamic parameter of the patient over the first time period to each of the plurality of reference waveforms of the hemodynamic parameter, each of the plurality of reference waveforms of the hemodynamic parameter corresponding to a respective sepsis value, and determining whether the patient is septic based on the sepsis value of the patient. The instructions may further be executed by the processor to display, on a display screen of a user device, at least one of the hemodynamic parameter of the patient and a determination of whether the patient is septic.

In a further aspect, a system for detecting sepsis in a patient is provided. The system includes one or more sensors configured to obtain physiological data from a patient, the physiological data comprising non-invasively obtained waveforms of physiological data, and a computer system in communication with the one or more sensors. The computer system may further include a processor, and a non-transitory computer readable medium in communication with the processor, the non-transitory computer readable medium having encoded thereon a set of instructions executable by the processor to perform various functions. The set of instructions may include instructions executable by the processor to obtain, with one or more sensors disposed in a sensor device, physiological data of a user continuously over a first time period, the physiological data comprising non-invasively obtained waveforms of physiological data, a hemodynamic parameter of the patient over the first time period, wherein the hemodynamic parameter is a patient-specific indication of the patient's proximity to hemodynamic decompensation at a given time, wherein the hemodynamic parameter is a numerical value indicating a relationship between an intravascular volume loss of a patient at the given time and an intravascular volume loss at hemodynamic decompensation of the patient. Determining the hemodynamic parameter of the patient includes applying a hemodynamic model to the physiological data, the hemodynamic model relating the physiological data to a value of the hemodynamic parameter, wherein the hemodynamic model comprises a plurality waveforms of reference data, comparing one or more waveforms of the physiological data of the patient to the each of the plurality of waveforms of reference data, each of the plurality of waveforms of reference data corresponding to a respective value of the hemodynamic parameter, and determining the hemodynamic parameter of the patient based on the comparison to each of the plurality of waveforms of reference data. The instructions may further be executable by the processor to determine based on the hemodynamic parameter of the patient over the first time period, whether the patient is septic. Determining whether the patient is septic further includes applying a sepsis model to the hemodynamic parameter of the patient over the first time period, wherein the hemodynamic parameter over the first time period is a waveform of the hemodynamic parameter of the patient, the sepsis model relating waveforms of the hemodynamic parameter to a sepsis value representing whether the patient is septic, wherein sepsis model comprises a plurality of reference waveforms of the hemodynamic parameter, comparing the waveform of the hemodynamic parameter of the patient over the first time period to each of the plurality of reference waveforms of the hemodynamic parameter, each of the plurality of reference waveforms of the hemodynamic parameter corresponding to a respective sepsis value, and determining whether the patient is septic based on the sepsis value of the patient. The instructions may further be executed by the processor to display, on a display screen of a user device, at least one of the hemodynamic parameter of the patient and a determination of whether the patient is septic.

While various aspects and features of certain embodiments have been summarized above, the following detailed description illustrates a few exemplary embodiments in further detail to enable one of skill in the art to practice such embodiments. The described examples are provided for illustrative purposes and are not intended to limit the scope of the invention.

In the following description, for the purposes of explanation, numerous specific details are set forth in order to provide a thorough understanding of the described embodiments. It will be apparent to one skilled in the art, however, that other embodiments may be practiced without some of these specific details. In other instances, certain structures and devices are shown in block diagram form. Several embodiments are described herein, and while various features are ascribed to different embodiments, it should be appreciated that the features described with respect to one embodiment may be incorporated with other embodiments as well. By the same token, however, no single feature or features of any described embodiment should be considered essential to every embodiment of the invention, as other embodiments of the invention may omit such features.

Unless otherwise indicated, all numbers used herein to express quantities, dimensions, and so forth used should be understood as being modified in all instances by the term “about.” In this application, the use of the singular includes the plural unless specifically stated otherwise, and use of the terms “and” and “or” means “and/or” unless otherwise indicated. Moreover, the use of the term “including,” as well as other forms, such as “includes” and “included,” should be considered non-exclusive. Also, terms such as “element” or “component” encompass both elements and components comprising one unit and elements and components that comprise more than one unit, unless specifically stated otherwise.

Various embodiments described herein, embodying software products and computer-performed methods, represent tangible, concrete improvements to existing technological areas, including, without limitation, medical diagnostic technology, medical monitoring technology, personal tracking technology, health monitoring technology, and/or the like. In other aspects, certain embodiments, can improve the functioning of the user equipment or systems themselves (e.g., personal trackers, health monitors, computer systems, etc.), for example, by enabling the detection and differentiation of sepsis in a patient through the collection, monitoring, and processing of non-invasively collected physiological signals from the patient.

To the extent any abstract concepts are present in the various embodiments, those concepts can be implemented as described herein by devices, software, systems, and methods that involve specific novel functionality (e.g., steps or operations), such as the detection and differentiation of sepsis from non-invasively collected physiological signals, and more specifically from a compensatory reserve index (CRI) value that is derived from non-invasively collected physiological signals.

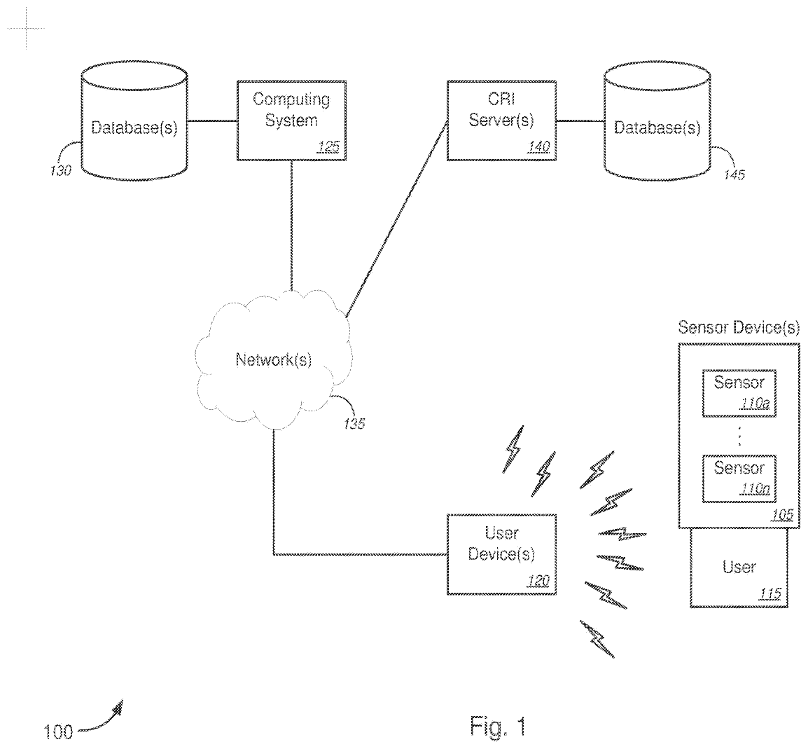

FIG. 1 illustrates a system 100 for detecting and differentiating sepsis, in accordance with various embodiments. The system 100 includes one or more sensor devices 105, which further include, without limitation, one or more sensors 110a-110n (collectively, “sensors 110”). The system further includes one or more user devices 120, computing system 125, one or more databases 130, one or more communication networks 135, one or more CRI servers 140, and one or more CRI databases 145. It should be noted that the various components of the system 100 are schematically illustrated in FIG. 1, and that modifications to the system 100 may be possible in accordance with various embodiments.

According to some embodiments, the one or more sensors 110 may include, without limitation, skin temperature sensors, electrodermal activity (EDA) sensors, thermometers, pulse oximeters, blood pressure (BP) sensors (including continuous BP monitors, blood pressure variability (BPV) monitors, a noninvasive blood pressure sensor such as the Nexfin (BMEYE, B.V.) or Finometer (Finapres Medical Systems B.V., etc.), respiration rate monitors, heart rate monitors (including continuous heart rate monitors, heart rate variability (HRV) monitors, etc.), fluid intake sensors, electrocardiographs, optical sensors (e.g., photodetectors in infrared (IR)/near-IR) as used in photoplethysmography (PPG), volume clamp, or other sensors suitable to capture waveforms generated during and/or by a cardiac cycle. Thus, the one or more sensors 110 may monitor, detect, collect or otherwise obtain waveform data generated by the patient during the cardiac cycle. The waveform data may include, for example, PPG waveforms, arterial and other pulsatile waveforms, ECG waveforms, blood pressure waveforms, respiratory rate waveforms, continuous oxygen saturation waveforms, or other suitable cardiological and other physiological data. The waveforms obtained by the one or more sensors 110 are herein referred to generally as physiological data.

In some embodiments, the one or more sensors 110 may further include, without limitation, accelerometers, gyroscopes, global navigation satellite system (GNSS) receivers, altimeters, pedometers, and/or other positional sensors. In some embodiments, the user device 120 and/or computing system 125 may be configured to mitigate motion artifacts from acquired waveform data (e.g., PPG, BP, etc.) based, at least in part, on motion data acquired from the one or more positional sensors. Motion artifacts, for example, may include noise introduced to the waveform data collected by the one or more sensors 110.

In some embodiments, the one or more sensors 110 may monitor at least one of the position and/or movements of the user 115, and may send data regarding the monitored at least one of the position and/or movements of the user to user device(s) 120 and/or computing system 125 (collectively, “computing system” or the like). In some instances, sending the data regarding the monitored at least one of the one or more position and/or movements of the user, and/or the like may comprise sending, with the one or more first sensors and to the computing system, data regarding the monitored at least one of the one or more postures or the one or more motions of the user, and/or the like, via wireless communications (as described above). Accordingly, in some embodiments, the motion artifacts may be mitigated from each of the respectively monitored pulsatile waveforms. Motion artifacts may include, for example, noise and/or error introduced in the pulsatile waveform by the user's movement. For example, in some embodiments, motion data from the one or more sensor devices 105 may be used to mitigate motion artifacts in the physiological data of the patient.

In some embodiments, the user device 120 may be configured to initiate sensor recording in the one or more sensor devices 105. The computing system may subsequently or concurrently store in database 130 an association between the initiated sensor recording of physiological data and any position and/or movement data. The one or more first sensors may subsequently send to the computing system 125 data regarding the monitored at least one of the positions and/or movements of the patient, where the physiological data of the user may include, but is not limited to, the data regarding the monitored at least one of the positions, movements of the patient.

In some cases, the one or more sensors 110 may be embodied outside of (or external to) the one or more sensor devices 105 (not shown). Alternatively, the one or more sensors 110 may each be encapsulated within a sensor device (e.g., sensor device 105 (as shown in FIG. 1), where each sensor device 105 may include, but is not limited to, one of a patch-based sensor device, a wristband-based sensor device, an armband-based sensor device, a headband-based sensor device, a belt-based sensor device, a leg strap-based sensor device, an ankle strap-based sensor device, or a shoe strap-based sensor device, and/or the like. In yet another alternative embodiment, a combination (not shown) of external sensors 110 (i.e., embodied external to sensor devices 105) and encapsulated sensors 110 (i.e., embedded within sensor devices 105) may be implemented. According to some embodiments, the sensor(s) 110 and/or the sensor device(s) 105 may be removably attached or affixed to a user 115. In some cases, the sensor(s) 110 and/or the sensor device(s) 105 may be removably attached or affixed to the user 115 via at least one of a patch, wristband, armband, headband, belt, leg strap, ankle strap, or shoestrap.

The system 100 further comprises one or more user devices 120. In some cases, the user device(s) 120 may each include, without limitation, a smart phone, smart watch, tablet computer, laptop computer, desktop computer, or dedicated sensor controller, and/or the like.

According to some embodiments, system 100 may further comprise a computing system 125 and corresponding database(s) 130 that are communicatively coupled to the user device(s) 120 and/or at least one of sensor device(s) 105, sensor(s) 110, and/or sensor(s) 125, via network(s) 135. In some embodiments, system 100 may further comprise CRI server(s) 140 and corresponding CRI database(s) 145 that may communicatively couple to at least one of the computing system 125, the user device(s) 120, the sensor device(s) 105, the sensor(s) 110, and/or the sensor(s) 125, via network(s) 135.

According to various embodiments, the user device(s) 120 may be communicatively coupled to each of at least one of network(s) 135, sensor device(s) 105, sensor(s) 110, and/or sensor(s) 125, via wireless communications systems or technologies (including, but not limited to, Bluetooth™ communications, such as Bluetooth Low Energy (“BTLE”), near field connection (“NFC”), Z-wave communications, ZigBee communications, XBee communications, or WiFi communications, and/or the like), as denoted in FIG. 1. In some cases, the network(s) 135 may include a local area network (“LAN”), including, without limitation, a fiber network, an Ethernet network, a Token-Ring™ network, and/or the like; a wide-area network (“WAN”); a wireless wide area network (“WWAN”); a virtual network, such as a virtual private network (“VPN”); the Internet; an intranet; an extranet; a public switched telephone network (“PSTN”); an infra-red network; a wireless network, including, without limitation, a network operating under any of the IEEE 802.11 suite of protocols, the Bluetooth™ protocol known in the art, the Z-Wave protocol known in the art, the ZigBee protocol or other IEEE 802.15.4 suite of protocols known in the art, and/or any other wireless protocol; and/or any combination of these and/or other networks. In a particular embodiment, the network may include an access network of the service provider (e.g., an Internet service provider (“ISP”)). In another embodiment, the network 135 may include a core network of the service provider, and/or the Internet.

In some aspects, the computing system (which may include the user device(s) 120 and/or the computing system 125, or the like) may receive physiological data of the user obtained by the one or more sensors 110 from the patient, as described above. The computing system 125 may analyze the received physiological data of the user 115 to detect and differentiate sepsis in the user 115, identify when a treatment is needed (such as fluid resuscitation and/or vasopressor administration), and the effectiveness of administered treatments. Thus, in some examples, the computing system 125 may determine at least one physiological state of the user 115, based at least in part on an analysis of at least one of the physiological data or CRI of the user 115.

In some embodiments, as described in greater detail below, CRI database(s) 145 may include one or more models of reference data, where the one or more models are generated empirically based on one or more test populations, each test population comprising a plurality of test subjects. Thus, the reference data may comprise data collected from the one or more test populations. In some examples, the one or more models may include, without limitation, a CRI model, sepsis model, fluid responsivity model, treatment effectiveness model, among other relevant models. The CRI model may relate reference physiological data gathered from a test population, such as BP waveforms, PPG waveforms, etc., to respective values of CRI. The sepsis model may, in turn, relate reference CRI waveforms and/or reference physiological data to the presence of sepsis, the severity of sepsis, and the effectiveness of treatments administered to treat the sepsis. In further embodiments, the one or more models may include individual-specific models, such as models specific to the user 115. Individual-specific models may be built from reference data (e.g., physiological data and/or reference CRI waveforms) obtained of the user 115 during a baseline (e.g., a normal/healthy) physiological state, or before the onset of sepsis.

In some embodiments, an artificial intelligence (AI)/machine learning (ML) algorithm may be employed to detect changes in the physiological data and/or CRI of a test subject and/or the user 115, and relate them to respective stages of sepsis. In this way, a model may be built relating reference data to, in this example, respective stages of sepsis. The AI/ML learning algorithm may be deployed, for example, in computing system 125 and/or CRI server(s) 140.

Similarly, an AI/ML algorithm may be employed at the user device 120, and may compare current (e.g., real-time) physiological data and/or CRI to reference data in the one or more models. Based on the comparison of the physiological data/CRI to the reference data, the model may then determine whether the patient is septic. The computing system 125 may then send the sepsis diagnosis and/or treatments to a user device 120 to be displayed to a user 115 and/or a medical provider treating the user 115.

In some embodiments, the physiological state determination may be performed in real-time or near-real-time based on the monitored sensor data (i.e., physiological data obtained by the one or more sensors 110 and/or CRI calculated based on the physiological data obtained in real-time).

Sepsis is often accompanied by the presentation of hemodynamic changes in the patient, such as hypovolemia, hypotension, and hyponatremia. The CRI of a patient is a potent and sensitive indicator of hemodynamic changes in a patient, and provides a measure of the relative hemodynamic state of a patient. For example, the CRI may be a sliding scale of CRI values, in which one or more ranges of CRI values correspond to respective states of hypovolemia (e.g., hemodynamic decompensation), euvolemia, and/or hypervolemia. Thus, in some embodiments, the CRI value of the patient may change over time as the physiological data of the patient changes over time. Sepsis and septic shock may then be detected based on the CRI value of the patient, and changes to the CRI value over time. For example, the CRI of the patient may itself be a continuous waveform and a function of time. A given CRI value of a patient may be a value of CRI at a given point time. The CRI may then be used to detect the presence of sepsis and/or septic shock, and to differentiate sepsis from localized infections. For example, changes in blood volume associated with the presence of sepsis are not always clinically apparent from the physiological data, such as BP (e.g., systolic BP), HR, temperature, oxygen saturation (SpO2), and respiratory rate (RR), as a patient's body is often able to compensate for blood volume loss by keeping these physiological measures within a normal range (e.g., maintaining homeostasis). Thus, CRI allows for earlier detection of sepsis within a patient before they are apparent in the physiological data. Furthermore, fluid resuscitation (e.g., fluid loading) is one of the primary treatments of sepsis as a way to increase blood volume and blood pressure. CRI similarly allows medical providers to assess whether the septic patient is fluid responsive, and measure the effectiveness of fluid resuscitation.

### Compensatory Reserve Index (CRI)

In various embodiments, CRI is determined by a novel algorithm, in which physiological data collected non-invasively from the patient may be used to determine the CRI value of the patient. CRI is also referred to herein and in the Related Applications as “cardiac reserve index” or “hemodynamic reserve index” (HDRI), all of which should be considered synonymous for purposes of this disclosure. While the term, “patient,” is used herein for convenience, that descriptor should not be considered limiting, because various embodiments can be employed both in a clinical setting and outside any clinical setting. Thus, the term, “patient,” as used herein, should be interpreted broadly and should be considered to be synonymous with “person.”

In some embodiments, CRI may be determined from waveform data (e.g., PPG waveforms) captured by the one or more sensors 110 from the patient (such as the one or more sensors 110 described above and the Related Applications, for example). The CRI may then be used to determine the presence of sepsis and the fluid responsiveness of sepsis in the patient. In other aspects, such functionality can be provided by and/or integrated with system 100, devices (such as sensor device(s) 105), tools, techniques, methods, and software described below and in the Related Applications.

As previously described, physiological data used to determine CRI of the patient may include, without limitation, PPG waveforms, other plethysmogram waveforms, arterial and other pulsatile waveforms, ECG waveforms, blood pressure waveforms, respiratory rate waveforms, continuous oxygen saturation waveforms, or other suitable cardiological and/or other physiological data.

The CRI of a patient represents a hemodynamic state of a patient relative to a state of hemodynamic decompensation (e.g., hypovolemia to the point where hemodynamic decompensation occurs, cardiovascular collapse, systolic blood pressure <70 mm Hg, etc.). Thus, the CRI indicates the hemodynamic state of a patient at a given time, where a range of CRI values corresponds to a range of hemodynamic states, ranging from the point of hemodynamic decompensation to euvolemia and/or a hypervolemic state.

For example, in various embodiments, CRI as a function of time “t” expresses the hemodynamic state of the patient as the relationship given by the following equation:

$$CRI ( t ) = 1 \frac{B L V ( t )}{B L {V}_{HDD}} \qquad ( Eq . 1 )$$

where BLV(t) is the intravascular volume loss (“BLV,” also referred to as “blood loss volume” in the Related Applications) of a person at time “t,” and BLV_{HDD} is the intravascular volume loss of a person when they enter hemodynamic decompensation (“HDD”). Hemodynamic decompensation is generally defined as occurring when the systolic blood pressure falls below 70 mmHg.

Accordingly, in the above equation, a CRI value of 1 corresponds to a state of euvolemia (e.g., a BLV(t) of 0 or no intravascular volume loss) and a CRI value of 0 corresponds to a level of hypovolemia at which hemodynamic decompensation occurs (e.g., a BLV(t) equal to BLV_{HDD}).

The level of intravascular volume loss is individual specific varies from person to person. Thus, in some embodiments, intravascular volume loss is modeled by the application of lower body negative pressure (LBNP), in which a linear or nonlinear relationship λ may be established with intravascular volume loss, as given by the following equation:

$$B L V = λ \cdot L B N P \qquad ( Eq . 2 )$$

Thus, LBNP can be used to model estimated the CRI for an individual undergoing a LBNP experiment as follows:

$$CRI = 1 - \frac{B L V ( t )}{B L {V}_{HDD}} \approx 1 - \frac{λ \cdot L B N P ( t )}{λ \cdot L B N {P}_{HDD}} = 1 - \frac{L B N P ( t )}{L B N {P}_{HDD}} \qquad ( Eq . 3 )$$

where LBNP(t) is the LBNP level that the individual is experiencing at time “t,” and, LBNP_{HDD} is the LNPB level that the individual will enter hemodynamic decompensation.

Thus, in various embodiments, a plurality of CRI models may be developed empirically from data collected from a test population comprising a plurality of test subjects. For example, in some embodiments, test subjects of the test population may be subjected to increasing levels of LBNP, until the onset of hemodynamic decompensation. Physiological data and beat-to-beat fluctuations in the physiological data may be collected at the various levels of LBNP. In some examples, blood pressure may be continuously collected from respective test subjects as LBNP is increased until the point of hemodynamic decompensation of the test subject. Thus, as described in the referenced related applications, the CRI model may be built based on physiological data collected from the test population, and relate the physiological data to the CRI value as follows:

$$CRI ( t ) = {f}_{CRI} ( {S}_{t} , F {V}_{t} ) \qquad ( Eq . 4 )$$

Where ƒ_{CRI}(Sₜ, FVₜ) is an algorithmic embodiment of the CRI model, representing the relationship of respective waveforms of physiological data, Sₜ, to a CRI value. In various embodiments, ƒ_{CRI}(Sₜ, FVₜ) is generated empirically, e.g., using the techniques described below, and/or in the Related Applications. FVₜ is a time history of fluid volume given to the patient (which can range from a single value to many hours of values), and Sₜ is a time history of raw sensor values, such as physiological data measured by the one or more sensors 110 (which can range from one value to many hours of values).

As described, the CRI model may relate physiological data, and the beat-to-beat variations in the physiological data, to relative CRIs. Thus, in various embodiments, the CRI model may comprise a plurality of waveforms of reference data (e.g., reference physiological data collected from the test population), where each of the waveforms corresponds to a respective CRI value. In some embodiments, one or more waveforms of reference data may correspond to the same (e.g., overlapping) values of CRI. Thus, the CRI of the patient may be estimated based on non-invasively collected physiological data from the patient.

Thus, CRI is a hemodynamic parameter that is indicative of an individual-specific proportion of intravascular fluid reserve remaining before the onset of hemodynamic decompensation. In some embodiments, CRI values may range from 1 to 0, where values near 1 are associated with euvovolemia (normal circulatory volume) and values near 0 are associated with the individual specific circulatory volume at which hemodynamic decompensation occurs. In other embodiments, other hemodynamic parameters may be used that are not limited in form to CRI. The hemodynamic parameters include, without limitation, any parameter indicating proximity of the patient to hemodynamic decompensation. Thus, the hemodynamic parameter may take a form different from the expression of CRI. For example, the hemodynamic parameter may indicate the relationship between the current intravascular volume loss of the patient and an intravascular volume loss of the patient at a point of hemodynamic decompensation and cardiovascular collapse. Thus, in some embodiments, a hemodynamic model may be derived empirically relating the physiological data to the hemodynamic parameters. Thus, with regard to the examples below, it is to be understood that a hemodynamic parameter may be used generically in place of the CRI, where CRI stands as one example of a hemodynamic parameter.

### CRI and Sepsis

Based on the estimated CRI value of the patient and/or changes to the CRI value of the patient over time, it may be determined whether the patient is septic. As with the CRI model, a septic state of the patient may be modeled by a sepsis model, in which the CRI values of a plurality of test subjects of a test population are collected at various stages of sepsis (e.g., no sepsis, mild sepsis, moderate sepsis, severe sepsis, and septic shock). For example, a patient with mild sepsis may exhibit only small deviations from normal, healthy baseline measures of CRI and/or underlying physiological data from which CRI may be determined. In contrast, a patient in septic shock may exhibit CRIs at or near the point of hemodynamic decompensation. Because the CRI values of a patient at different stages of sepsis vary from individual to individual, the sepsis model may relate changes in the CRI values over time of the patient to a stage of sepsis for that patient based on a plurality of CRI waveforms collected from the test population. Thus, like in the CRI model, the sepsis model may itself comprise a plurality of reference CRI waveforms (e.g., reference data), where each CRI waveform may correspond to a respective stage of sepsis. In some embodiments, one or more reference CRI waveforms (e.g., reference data) may correspond to the same (e.g., overlapping) stages of sepsis. Thus, the CRI of the patient may be estimated based on non-invasively collected physiological data from the patient.

Accordingly, the sepsis model may be empirically generated based on a test population from which physiological data and/or CRI is continuously measured at the various stages of sepsis. Alternatively, in some embodiments, the sepsis model may be generated based on empirical data relating physiological data and/or intravascular volume loss to the various states of sepsis. Thus, using CRI and/or physiological data, sepsis may be detected.

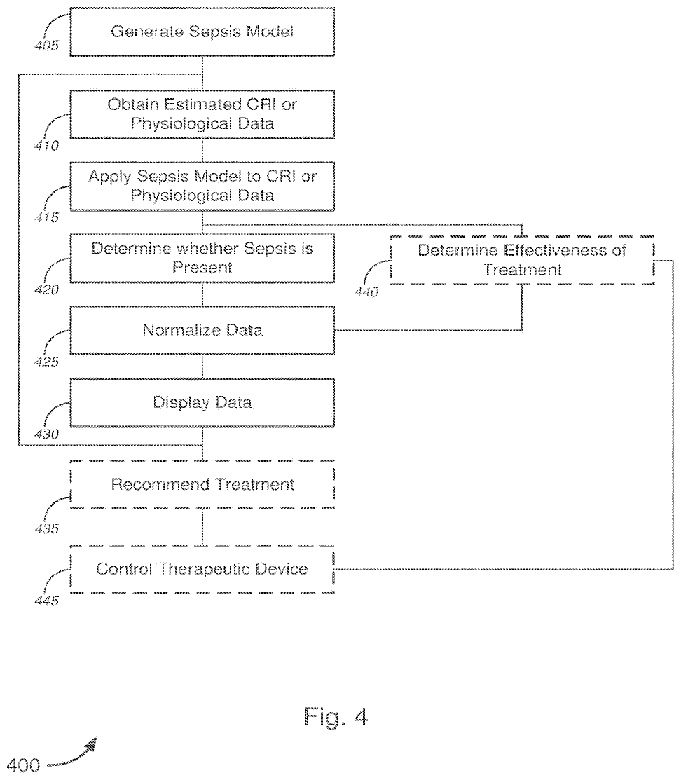

In some embodiment, the stage of sepsis (“SPS”) can be expressed as a value between 0 and 1; an SPS=1 may correspond to a state of septic shock, whereas an SPS=0, corresponds to no sepsis, and when SPS is a value between 1 and 0, the value is indicative of a stage of sepsis before septic shock sets in (e.g., mild sepsis, moderate sepsis, and severe sepsis). It is to be understood that in other embodiments, the range and scaling of SPS values may be configured differently. For example, in alternative embodiments, the SPS may range in value arbitrarily, for example, between 10 and 0, 100 and 0, etc. Furthermore, the scale of SPS values may correspond to the stages of sepsis in a non-linear manner, or bear an inverse relationship to sepsis (e.g., 0 corresponding to septic shock, and 1 corresponding to no sepsis). In some further embodiments, SPS values may correspond to a confidence level as to the presence of sepsis in the patient. In an aspect of some embodiments, a general expression for the detection of sepsis is as follows:

$$S P S = {S}_{SPS} ( {CRI}_{t} , F {V}_{t} , {S}_{t} ) \qquad ( Eq . 5 )$$

where ƒ_{SPS}(CRIₜ, FVₜ, Sₜ) is an algorithmic embodiment of the sepsis model, relating CRI to SPS. In various embodiments, ƒ_{SPS}(CRIₜ, FVₜ, Sₜ) is generated empirically, e.g., using the techniques described with respect to FIG. 4 below, and/or in the Related Applications. CRIₜ is a time history of CRI values (e.g., a CRI waveform over time), which can range from a single CRI value to many hours of CRI values. FVₜ is a time history of fluid volume given to the patient (which can range from a single value to many hours of values), and Sₜ is a time history of raw sensor values, such as physiological data measured by the one or more sensors 110 (which can range from one value to many hours of values).

The functional form of Eq. 5 is similar to but not limited to the form of the SPS model in the sense that time histories of (CRIₜ, FVₜ, Sₜ) data gathered from human subjects at various levels of sepsis are compared to time histories of (CRIₜ, FVₜ, Sₜ) for the current patient being monitored. The estimated SPS for the current patient is then that which is the closest in (CRIₜ, FVₜ, Sₜ) space to the previously gathered data.

While Eq. 5 is the general expression for SPS, various embodiments may use subsets of the parameters considered in Eq. 5. For instance, in one embodiment, a model may consider only the volume of fluid and CRI data, without accounting for raw sensor input. In that case, SPS can be calculated as follows:

$$S P S = {S}_{SPS} ( {CRI}_{t} , F {V}_{t} ) . \qquad ( Eq . 6 )$$

Similarly, some models may estimate SPS based on sensor data (e.g., physiological data obtained from the patient), rather than first estimating CRI, in which case, SPS can be expressed as:

$$S P S = {f}_{SPS} ( F {V}_{t} , {S}_{t} ) . \qquad ( Eq . 7 )$$

The choice of parameters to use in modeling SPS is discretionary, and it can depend on what parameters are shown (e.g., using the techniques of FIG. 4, below) to result in the best prediction of SPS.

Moreover, the sepsis model ƒ_{SPS} may differentiate sepsis from other forms of localized infection, based on the input parameters (CRIₜ, Sₜ), and variations in the input parameters as given by the model ƒ_{SPS}. In some embodiments, the SPS model may further include physiological data and/or CRI collected from test subjects of a test population that are not septic, but have other forms of localized infection. Thus, in some embodiments, ƒ_{SPS} may differentiate sepsis from other forms of infection by identifying that the patient is suffering from another form of localized infection, by identifying that the patient is not suffering from sepsis, or both identifying that the patient is not suffering from sepsis and also suffering from a localized infection.

In another aspect, the effectiveness of treatments, such as fluid loading/resuscitation, administration of vasopressors, etc. In some embodiments, the effectiveness of fluid resuscitation (e.g., fluid responsivity of sepsis) may be assessed. For example, in some embodiments, the effectiveness of fluid resuscitation may be estimated by predicting the volume, V, of fluid necessary for effective hydration of the patient. This volume, V, can indicate a volume of fluid needed to maintain a threshold intravascular volume (e.g., euvolemia, or a minimum acceptable level of intravascular volume). Like SPS, the value of V can be estimated/predicted using the modeling techniques described herein and in the Related Applications. In a general case, V can be expressed as the following:

$$V = {f}_{V} ( {CRI}_{t} , F {V}_{t} , {S}_{t} ) \qquad ( Eq . 8 )$$

where V is an estimated volume of fluid needed by a patient need to prevent over or under hydration, ƒ_{V}(CRIₜ, FVₜ, Sₜ) is an algorithm embodied by a model (e.g., a fluid responsivity model) generated empirically, e.g., using the techniques described with respect to FIG. 4 below, and/or in the Related Applications, CRIₜ is a time history of CRI values, FVₜ is a time history of fluid volume given to the patient (e.g., one or more of bolus volume, IV flow/drip rate, etc.), and Sₜ is a time history of physiological data received from the one or more sensors.

As with the estimate of SPS, various embodiments can employ subsets of the parameters used in the general expression of Eq. 8. Thus, different embodiments may calculate V as follows:

$$V = {f}_{V} ( {CRI}_{t} , F {V}_{t} ) \qquad ( Eq . 9 )$$

$$or$$

$$V = {f}_{V} ( F {V}_{t} , {S}_{t} ) . \qquad ( Eq . 10 )$$

Yet another way of assessing effectiveness of fluid resuscitation is estimating the probability P_{ƒ} that the patient requires additional fluid. For example, the probability P_{ƒ} may estimate the likelihood that the patient requires fluid to be administered. The value of this probability, which can be expressed, e.g., as a percentage, as a decimal value between 0 and 1, etc. may be estimated using the following expression:

$${P}_{f} = {f}_{{P}_{f}} ( {CRI}_{t} , {S}_{t} ) \qquad ( Eq . 11 )$$

where P_{ƒ} is the estimated probability that the patient requires fluid, ƒ_{P}_{_{ƒ}}(CRIₜ, Sₜ) is a relationship derived based on empirical study, CRIₜ is a time history of CRI values, and Sₜ is a time history of physiological data received from the one or more sensors. Once again, this general expression can be employed, in various embodiments, using subsets of the parameters in the general expression, such as the following:

$${P}_{f} = {f}_{{P}_{f}} ( {CRI}_{t} ) \qquad ( Eq . 12 )$$

$$or$$

$${P}_{f} = {f}_{{P}_{f}} ( {S}_{t} ) . \qquad ( Eq . 13 )$$

In the estimate of any of SPS, V, or P_{ƒ}, the function ƒ expresses a relationship that is derived based on empirical study of data gathered from a test population. In a set of embodiments, for example, various sensor data can be collected from test subjects of the test population before, during, and/or after fluid has been administered to a septic patient, a dehydrated patient with other form of localized infection, or under other conditions that may simulate such situations.

In yet further embodiments, a model may be built to assess similarly whether, and a dosage of vasopressor (D_{VP}) or other therapeutic agent should be administered, and whether the vasopressor is working effectively. For example, the determination of whether a vasopressor should be administered may be expressed as a numeric confidence level (P_{VP}). In some examples, the determination of whether the vasopressor is working effective may be determined as a confidence level (P_{effVP}) that the patient is maintaining an intravascular volume and/or pulsatile pressure. As with the estimation of CRI, SPS, and effectiveness of fluid resuscitation, a determination of treatment effectiveness values (e.g., D_{VP}, P_{VP}, and P_{effVP}) may be determined from respective empirically generated treatment effectiveness models (e.g., ƒ_{DVP}, ƒ_{PVP}, ƒ_{PeffVP}) relating CRI and/or physiological data to D_{VP}, P_{VP}, and P_{effVP}, respectively, as given by the following expressions:

$${D}_{VP} = {f}_{{D}_{VP}} ( {CRI}_{t} ) \qquad ( Eq . 14 )$$

$${P}_{VP} = {f}_{{P}_{VP}} ( {CRI}_{t} ) \qquad ( Eq . 15 )$$

$$and$$

$${P}_{{P}_{effVP}} = {f}_{{P}_{effVP}} ( {CRI}_{t} ) \qquad ( Eq . 16 )$$

As with CRI, SPS, and fluid resuscitation, the various empirically generated models (ƒ_{DVP}, ƒ_{PVP}, ƒ_{PeffVP}) may alternatively be written as functions of additional or fewer parameters, or different combinations of parameters, such as FVₜ and Sₜ.

Thus, measures of CRI, SPS, V, P_{ƒ}, D_{VP}, P_{VP}, and/or P_{effVP} may be used for the early detection of sepsis before it is clinically apparent in conventional physiological signals, such as heart rate, blood pressure, body temperature, etc. A severity of sepsis in a patient may also be determined on an individualized basis. Moreover, sepsis may be differentiated from other forms of infection. A determination to provide fluid resuscitation (e.g., fluid loading), and the effectiveness of such fluid resuscitation, and in turn the fluid responsivity of the septic patient, may also be determined. As previously described, fluid loading is often one of the first treatments to severe cases of sepsis and/or septic shock. Oftentimes, however, fluid loading can be harmful or otherwise detrimental to a patient, and especially so when the sepsis is not responsive to fluid resuscitation. Thus, a determination as to whether the patient is fluid responsive may be used to determine whether fluid loading should be ceased or should be continued. A determination as to whether and how to administer other treatments, such as vasopressors, and/or antibiotics may also be determined, as well as a severity of the sepsis in a patient. Accordingly, the tools and techniques for estimating and/or predicting CRI can have a variety of applications in a clinical setting, including, without limitation, the diagnosis and treatment of sepsis.

Moreover, CRI allows for the above capabilities through the use of non-invasively collected physiological data. For example, conventional approaches may rely on the analyses of traditional vital signs and mining of electrical health records for vital sign entries that match known criteria for early sepsis (sepsis alerts), rather than innovating completely new information sources. Additional investigators are focused on the use of biomarkers (e.g., PCT, CRP) or nanotechnology for diagnosis and assessing severity of sepsis. Finally, the SOFA (more of an epidemiologic and research tool than a clinical one) and qSOFA scores have been widely used—but these rely on vital signs as well as laboratory tests (SOFA) or vital signs alone (qSOFA). None of these techniques provide immediate, inexpensive, sensitive bedside physiology based diagnostic and monitoring capability for sepsis. Moreover, conventional approaches to assessing the effectiveness of fluid resuscitation rely on parameters, such as pulse pressure variation, stroke volume, and/or pulse pressure index. These techniques require the invasive collection of the required parameters, such as intubation and indwelling arterial lines, presenting significant limits to the ease of use and wide adoption of such techniques.

Accordingly, the system 100 identifies and differentiates septic patients based on changes exhibited in the CRI of a patient, which may be determined from physiological data that is collected non-invasively. In some embodiments, profiles of the CRI (as compared with base measurements of CRI of the individual patient or a compilation of measurements of reference CRI waveforms (e.g., reference data) across a sample of multiple test subjects) may be indicative of health, fitness, and/or other physiological states of the user. In such cases, a CRI server or other computational device may monitor physiological data of the user (e.g., by using sensors, including, but not limited to the one or more sensors 110, as described herein) to estimate a CRI of the patient, and may further analyze the estimated CRI to detect and differentiate sepsis in the patient, and an effectiveness of a treatment, such as fluid resuscitation and vasopressor administration. In differentiating sepsis, the CRI of the patient, and particularly how the CRI of the patient changes over time, may be used to determine a level of tolerance to liquid limitations of the patient, a state of dehydration of the patient, a level of tolerance to blood loss of the user, one or more states of illness of the user (including, but not limited to, sepsis, flu, cold, viral infection, bacterial infection, or other localized infection, heart disease, and/or the like). Such physiological states may then be presented to the user (or a physician or other healthcare provider of the user) using a user interface of a user device, a display screen of a user device, a web portal, a software application (“app”), and/or the like.

Thus, in various embodiments, the one or more sensors 110 may obtain physiological data non-invasively. A set of embodiments provides methods, systems, and software that can be used, in many cases noninvasively, to quickly and accurately detect and differentiate sepsis in a patient, and further to assess the fluid responsivity of sepsis in the patient from the non-invasively collected physiological data. In various embodiments, a number of different physiological data may be obtained from the patient, and the analysis of the physiological data may vary according to which specific physiological parameters/waveforms are measured (and which, according to the generated model, are found to be most predictive of sepsis or the effectiveness of a treatment such as fluid resuscitation or vasopressors). In some cases, the physiological data (e.g., continuous waveform data captured by a photoplethysmograph) may be used to directly detect and differentiate sepsis, and determine the effectiveness of a treatment (e.g., fluid resuscitation, vasopressors, etc.). In yet other cases, both CRI and certain physiological data (which may or may not have been used to determine the CRI), may be used together to make such determinations as to the detection, differentiation, and treatment of sepsis.

In various embodiments, CRI, SPS, and measures of the effectiveness of treatments (such as fluid resuscitation and/or vasopressors: V, P_{ƒ}, D_{VP}, P_{VP}, and/or P_{effVP}) may be determined based on (i) a fixed time history of patient monitoring of physiological data (for example a 30 second or 30 heart beat window); (ii) a dynamic time history of patient monitoring of physiological data (for example monitoring for 200 minutes, the system may use all sensor information gathered during that time to refine and improve CRI estimates, hydration effectiveness assessments, etc.); (iii) established baseline estimates when the patient is normovolemic (no volume loss has occurred); and/or (iv) no baseline estimates when the patient is normovolemic.

In some embodiments, the system may also recommend and/or control treatments, based on the CRI of the patient. For example, treatment options can include, without limitation, such things as optimizing hemodynamics, administering fluids (e.g., fluid loading/fluid resuscitation), adjustments to fluid administration (e.g., controlling the flow rate of an IV pump or the drip rate of an IV drip, adjusting the volume of a bolus), administering vasopressors, and administering antimicrobials.

Thus, the system 100 provides accurate and sensitive diagnosis, patient monitoring, treatment planning, treatment monitoring, and therapeutic control functionalities, functionalities which include, but are not limited to:
(A) Prediction of the development of sepsis/septic shock, and the progression of sepsis, in patients who present with signs and symptoms of infection.
(B) An assessment of the severity of disease superior to any other noninvasive metric now available.
(C) Real time, continuous monitoring of the efficacy of treatments including but not limited to fluid administration and use of vasopressors
(D) Post acute monitoring—once patients are stabilized the system 100 allows continuous real-time monitoring for relapse and/or worsening sepsis
(E) Assess whether patients are in an early hyper inflammatory or a more protracted immunosuppressive phase of sepsis
(F) Differentiation of sepsis from localized infection
(G) Stratification of risk allowing for precision and personalized intervention
(H) Optimization of fluid administration
(I) Optimization of pressor administration
(J) Determination of the need for vasopressors
(K) Real time measurement of proximity to vascular collapse
(L) Improved function of existing sepsis alert systems by incorporation into existing algorithms
(M) Differentiation of sepsis from dehydration in the presence of localized infection
(N) Use as a triage tool to determine who is “sickest”
(O) Monitor for deterioration of patients with known sepsis
(P) Use as a syndromic surveillance system in public health settings to identify outbreak of disease.
(Q) Recommend targeted antimicrobial therapy in localized infection to avoid overuse of broad spectrum antibiotics in populations with suspected sepsis—with attendant implications for antibiotic resistance.
(R) Integrate with other AI/ML techniques used for sepsis alerts

According to some embodiments, implementation of software and algorithms (which may be performed on the user device(s) 120, the computing system 125, CRI server(s) 140, or other computational device(s)) may include, without limitation, (A) methodology for mapping physiological data to sepsis and/or a severity of sepsis—the algorithmic method for performing such mapping including, but not limited to, deep learning, clustering unsupervised and semi-supervised algorithms, principle component analysis and related linear and non-linear techniques such as independent component analysis and network component analysis, Mahalanobis distance and Polynomial, minimum (or percent of) and maximum (or percent of) sensor readings during relevant time intervals, supervised learning techniques, or probabilistic methods yielding estimates of confidence, and/or the like; (B) methodology for mapping the results of characterized recording timelines in (A) above to status and/or prediction of future status—the algorithmic method for performing such mapping including, but not limited to, deep learning, supervised learning techniques, or probabilistic methods yielding estimates of confidence, and/or the like; or (C) methodology for mapping the results of characterized recording timelines in (B) above to status and/or prediction of future status—the algorithmic method for performing such mapping including, but not limited to, deep learning, supervised learning techniques, or probabilistic methods yielding estimates of confidence, and/or the like.

Alternatively, implementation of software and algorithms (which may be performed on the user device(s) 120, the computing system 125, or other computational device(s)) may include, without limitation, (D) methodology for mapping one or more recorded timelines of CRI to the presence of sepsis and/or severity of sepsis in the patient—the algorithmic method for performing such mapping including, but not limited to, deep learning, clustering unsupervised and semi-supervised algorithms, principle component analysis and related linear and non-linear techniques such as independent component analysis and network component analysis, Mahalanobis distance and Polynomial Mahalanobis Distance Metric, or minimum (or percent of) and maximum (or percent of) sensor readings during relevant time intervals, and/or the like; or (E) methodology for mapping the results of characterized recording timelines in (D) above to status and/or prediction of future status—the algorithmic method for performing such mapping including, but not limited to, deep learning, supervised learning techniques, or probabilistic methods yielding estimates of confidence, and/or the like.

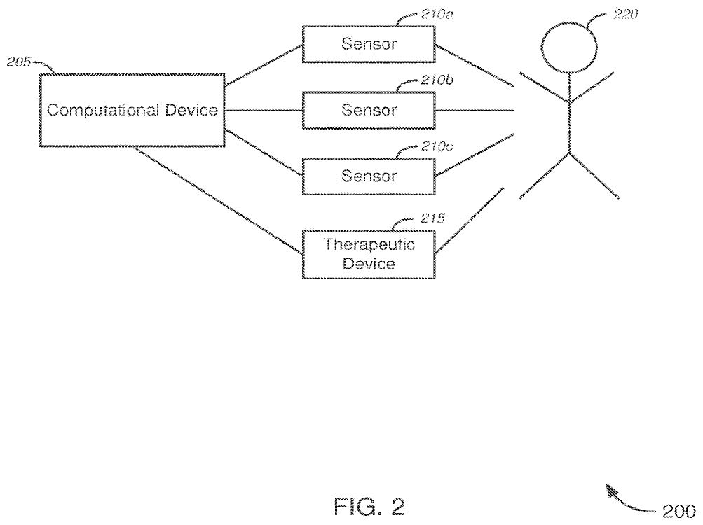

These and other functions of the system 100 (and its components) are described in greater detail below with respect to FIGS. 2-4.

FIG. 2 is a schematic diagram illustrating a system 200 for estimating compensatory reserve, which can be used for implement sepsis detection and differentiation, in accordance with various embodiments. The system 200 includes a computer system or computational device 205 in communication with one or more sensors 210 (which may include sensors 210a, 210b, and 210c, or the like), each of which may be configured to obtain physiological data from the patient 220. The computer system 205 may be any system of one or more computers that are capable of performing the techniques described herein. In a particular embodiment, for example, the computer system 205 is capable of reading values from the sensors 210; generating models of physiological state from those sensors; employing such models to make individual-specific estimations, predictions, or other diagnoses; displaying the results; recommending and/or implementing a therapeutic treatment as a result of the analysis; and/or archiving (learning) these results for use in future, model building and predictions; or the like.

The sensors 210 can be any of a variety of sensors (including, without limitation, those described herein) for obtaining physiological data from the subject. An exemplary sensor suite may include a Finometer sensor for obtaining a noninvasive continuous blood pressure waveform, a pulse oximeter sensor, an Analog to Digital Board (National Instruments USB-9215A 16-Bit, 4 channel) for connecting the sensors (either the pulse oximeter and/or the finometer) to the computer system 205. More generally, in an embodiment, one or more sensors 210 may obtain, e.g., using one or more of the techniques described herein, continuous physiological waveform data, such as continuous blood pressure. Input from the sensors 210 can constitute continuous data signals and/or outcomes that can be used to generate, and/or can be applied to, a predictive model as described below.

Merely by way of example, the one or more sensors 210 may further include, without limitation, at least one of one or more accelerometers, one or more gyroscopes, one or more location sensors, one or more pedometers, or one or more altimeters, and/or the like. Alternatively, or additionally, the one or more sensors 210 may include, but are not limited to, at least one of one or more skin temperature sensors; one or more moisture sensors; one or more resistance sensors; one or more electrodermal activity (“EDA”) sensors; one or more body temperature sensors; one or more core temperature sensors; one or more fluid intake measurement sensors; one or more sensors measuring a CRI of the patient; one or more sensors measuring hemodynamic status of the patient; one or more sensors measuring closeness of hemodynamic collapse due to at least one of heat stress, hydration, or central fluid loss; one or more sensors that continuously capture one or more pulsatile components of a cardiac cycle of the user; one or more electrocardiograph sensors; or one or more respiration rate sensors; and/or the like. In some instances, the one or more sensors that continuously capture the one or more pulsatile components of the cardiac cycle of the user may include, without limitation, at least one of radio frequency (“RF”) sensor, a photoplethysmograph (“PPG”), a volume clamp, or a continuous blood pressure (“BP”) sensor, and/or the like.

In some cases, the structure or system may include a therapeutic device 215 (also referred to herein as a “physiological assistive device”), which can be controlled by the computer system 205 to administer therapeutic treatment, in accordance with the recommendations developed by analysis of a patient's physiological data. In a particular embodiment, the therapeutic device 215 may comprise an IV drip, infusion pump, or valve, which can be controlled by the computer system 205 based on the estimated CRI of the patient, as described in further detail below. Further examples of therapeutic devices 215 in other embodiments can include a cardiac assist device, hemodialysis machine, ventilator, an automatic implantable cardioverter defibrillator (“AICD”), pacemakers, an extracorporeal membrane oxygenation circuit, a positive airway pressure (“PAP”) device (including, without limitation, a continuous positive airway pressure (“cPAP”) device, or the like), an anesthesia machine, an integrated critical care system, a medical robot, intravenous and/or intra-arterial pumps that can provide fluids and/or therapeutic compounds (e.g., through intravenous injection), a heating/cooling blanket, and/or the like.

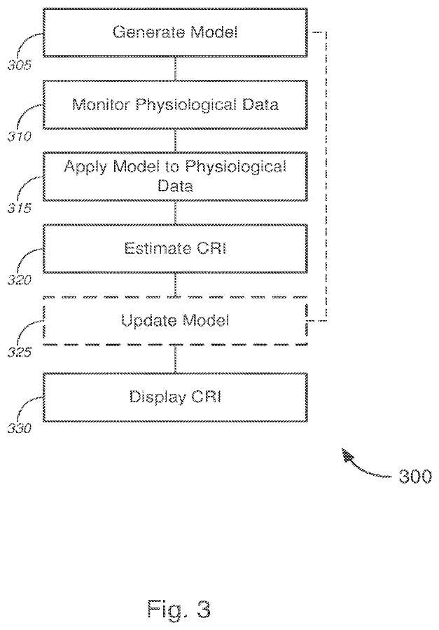

System 200 of FIG. 2 may otherwise be implemented in a similar manner as described in detail herein with respect to system 100 of FIG. 1, method 300 of FIG. 3, and/or method 400 of FIG. 4.

FIG. 3 is a flow diagram illustrating a method 300 of estimating a patient's compensatory reserve, in accordance with various embodiments. The method 300 may comprise, at block 305, generating a model, e.g., with a computer system, against which physiological data may be analyzed and compared to estimate and/or predict a CRI of the patient. In a general sense, generating the model may comprise receiving data pertaining to physiological data of a patient and/or from a plurality of test subjects of a test population, to obtain a plurality of physiological data sets. Such data can include PPG waveform data, BP waveform data, and/or any other type of sensor data including, without limitation, data captured by sensors described herein and in the Related Applications.

Generating the model may further comprise directly measuring one or more waveforms of reference data from respective test subjects while the test subjects are subjected to various levels of simulated intravascular volume loss (e.g., through the application of lower body negative pressure (LBNP)), or during actual intravascular volume loss (e.g., due to illness, trauma, etc.).

In some embodiments, generating the model may further comprise correlating the CRI with the measured reference data. Thus, reference data collected during the respective volumes of intravascular volume loss, simulated or otherwise, may be associated with respective CRI values associated with the respective volumes of intravascular volume loss. A variety of techniques may be employed to generate a model in accordance with different embodiments. One exemplary technique for generating a model of CRI may include using a machine-learning algorithm to optimize the correlation between measured reference data (such as PPG waveform data, to name one example) and intravascular volume loss and/or CRI derived from intravascular volume loss. It should be appreciated, however, that any suitable technique or model may be employed in accordance with various embodiments.

The method 300 further includes, at block 310, monitoring physiological data of the patient with one or more sensors. As previously described, a variety of physical parameters can be monitored non-invasively, depending on the nature of the anticipated physiological state of the patient. In an aspect, monitoring the physiological data might comprise receiving, e.g., from a physiological sensor, continuous waveform data, which may be sampled as necessary. Such data may include, without limitation, PPG waveform data (such as that generated by a pulse oximeter), blood pressure data, or any other pulsatile data generated by the patient during a cardiac cycle. Thus, physiological data may be gathered in real-time or near-real time from the patient, and analyzed accordingly.

The method 300 further comprises applying the model to the physiological data. In various embodiments, the physiological data may be analyzed, with a computer system (e.g., the system 100 and/or system 200 above), and the model applied to the physiological data. In some examples, one or more waveforms of the physiological data may be compared against one or more waveforms of the reference data in the model. Thus, the model may be applied to the physiological data, which may yield a corresponding CRI value based on an analysis and/or comparison of the physiological data against the reference data.

Merely by way of example, in some cases, sensor data (e.g., physiological data) may be analyzed directly against a generated model. For example, respective waveform data of the physiological data may be sampled (e.g., any of the data described herein and in the Related Applications, including, without limitation, arterial waveform data, such as continuous PPG waveforms and/or continuous noninvasive blood pressure waveforms) for a specified period, such as 30 heartbeats. That sample may be compared with a plurality of waveforms of reference data corresponding to CRI values. As described above, the waveforms of reference data are derived as part of the model developed using the algorithms described in this and the Related Applications, as the result of experimental data (e.g., from a test population), or from baseline measurements obtained from the patient.

The method 300 further includes, at block 320, estimating the CRI value of the patient. In some embodiments, the sampled waveform of physiological data may be compared with a plurality of reference waveforms corresponding to a range of respective CRI values. Any number of sampled waveforms of physiological data may be used for the comparison; for example, if there is a nonlinear relationship between the measured physiological data and the CRI values, more sample waveforms may provide for a better comparison. From the comparison, a similarity coefficient may be calculated (e.g., using a least squares or similar analysis) to express the similarity between the sampled waveform and each of the reference waveforms. These similarity coefficients may be used to normalized and/or weight a CRI value corresponding to the respective waveform of reference data, and the CRI values as normalized/weighted by the similarity coefficients may be summed to produce an estimated CRI value of the patient.

The method 300 might further comprise normalizing the results of the analysis (block 335), such as the compensatory reserve, dehydration state, and/or probability of bleeding, to name a few examples. Merely by way of example, the estimated/predicted compensatory reserve of the patient can be normalized relative to a normative normal blood volume value corresponding to euvolemia, a normative excess blood volume value corresponding to circulatory overload, and a normative minimum blood volume value corresponding to cardiovascular collapse. Any values can be selected as the normative values. Merely by way of example, in some embodiments, the normative excess blood volume value may be >1, the normative normal blood volume value may be 1, and the normative minimum blood volume value may be 0. As an alternative, in other embodiments, the normative excess blood volume value might be defined as 1, the normative normal blood volume value might be defined as 0, and the normative minimum blood volume value at the point of cardiovascular collapse might be defined as −1. As can be seen from these examples, different embodiments might use a number of different scales to normalize CRI and other estimated parameters.

The estimated CRI of the patient may, in some embodiments, be based on several factors. Merely by way of example, in some examples, the estimated CRI value may be based on a fixed time history of monitoring the physiological data of the patient and/or a dynamic time history of monitoring the physiological data of the patient. In other examples, the estimated CRI value may be based on a baseline estimate of the patient's CRI established when the patient is euvolemic. In still other cases, the estimate might not be based on a baseline estimate of the patient's CRI established when the patient is euvolemic, but rather based on a baseline estimate of the patient's CRI established when the patient is in another physiological state or condition (e.g., localized infection, no infection, dehydrated state with localized infection, dehydrated with no infection, etc.).

In some embodiments, the method 300 further includes, at optional block 325, updating the model with the physiological data obtained in real-time from the patient. In some embodiments, an intravascular volume loss (BLV(t)) may be measured, retrospectively or in real-time, and physiological data obtained from the patient, as described in block 310, may be associated with a respective CRI value, and the model updated to reflect the association. In other words, the physiological data obtained from the patient in real-time may be used as reference data for a future estimate of CRI.

At block 330, the method 300 may continue by displaying the estimated CRI value in real-time. As previously described, in this and the above referenced related applications, a display device may be configured to display the estimated CRI value. In some embodiments, a normalized value of CRI may be displayed, where an estimate of “0” indicates that the patient is at a point of hemodynamic collapse, and “1” indicates a state of euvolemia. In further embodiments, CRI may be displayed as and/or along with a “fuel gauge” type bar graph to quickly convey, via color coding (e.g., red corresponding to a lower range of CRI values, yellow corresponding to a range of values between red and green, and green corresponding to a higher range of CRI values), a range within which the CRI value falls, and the risk/danger to the patient.

FIG. 4 is a flow diagram illustrating a method 400 of determining whether a patient is septic and differentiating sepsis from other conditions, in accordance with various embodiments. The method 400 begins, at block 405, by generating a sepsis model. As described above with respect to the CRI model, the sepsis model may be generated empirically, based on reference data. Reference data may include reference physiological data collected empirically from a test population comprising a plurality of test subjects. Alternatively, the reference data may include historic physiological data collected from the patient.

Thus, generating the model may include directly obtaining one or more reference CRI waveforms from respective test subjects (e.g., calculated from reference data) while the test subjects have sepsis, and as sepsis progresses in a test subject. Thus, in some embodiments, generating the model may further comprise correlating the reference CRI waveforms with the presence of sepsis, the severity of sepsis, and the effectiveness of a treatment of sepsis. Reference CRI waveforms collected during the respective stages of sepsis in respective test subjects may be associated with respective CRI reference waveforms. A variety of techniques may be employed to generate a model in accordance with different embodiments. One exemplary technique for generating a sepsis model may include using a machine-learning algorithm to optimize the correlation between measured reference data (such as PPG waveform data, intravascular volume loss and/or CRI derived from intravascular volume loss) and sepsis and/or effectiveness of treatment, etc. It should be appreciated, however, that any suitable technique or model may be employed in accordance with various embodiments.

The method further includes, at block 410, obtaining estimated CRI and/or physiological data from the patient. For example, in some embodiments, the physiological data from the patient may be obtained in real-time (or near real-time) from one or more sensors. The physiological data may then be used to estimate a CRI of the patient over time, as described above with respect to the method 300. At block 415, the method 400 continues by applying the sepsis model to the estimated CRI and/or physiological data. As previously described with respect to the CRI model, the sepsis model may similarly be applied to the patient's estimated CRI and/or physiological data.

In various embodiments, the physiological data may be analyzed, with a computer system (e.g., the system 100 and/or system 200 above) to produce a CRI. The CRI of the patient may then, similarly be analyzed and the model applied to the CRI. In some examples, one or more waveforms of CRI, which may vary over time, may be compared against one or more reference CRI waveforms of the sepsis model.

At block 420, the method 400 continues by determining whether sepsis is present, and at optional block 440, by determining an effectiveness of treatment in the patient. Thus, the model may be applied to the CRI, which may yield a corresponding determination of sepsis, such as SPS described above, among other parameters, with respect to FIG. 1. Thus, CRI may be used to determine the presence of sepsis, a severity of sepsis, and/or the effectiveness of treatments administered to treat the sepsis. In further embodiments, physiological data obtained from the patient may also be used instead, or in addition to, CRI. In further examples, the sepsis model may similarly include waveforms of reference physiological data which may be applied to real-time (or near real-time) physiological data obtained from the patient, and determinations of the presence of sepsis, severity of sepsis, and/or the effectiveness of treatments. Treatments may include, without limitation, fluid resuscitation and/or administration of vasopressors.

At block 425, the method 400 further includes normalizing the data produced by the analysis of the data (e.g., the determination of sepsis and/or effectiveness of treatment). For example, in some embodiments, merely by way of example, sensor data (e.g., physiological data) and/or estimated CRI waveforms may be analyzed directly against a generated sepsis model. For example, respective waveform data (e.g., physiological data and/or estimated CRI) may be sampled (e.g., any of the data described herein) for a specified period, such as 30 heartbeats. The sample may be compared with a plurality of reference CRI waveforms corresponding to CRI values. As described above, the reference CRI waveforms may be derived as part of the model, developed using the algorithms described in this and the Related Applications, as the result of experimental data (e.g., from a test population), or from baseline measurements obtained from the patient.

Any number of sampled waveforms of CRI and/or physiological data may be used for the comparison; for example, if there is a nonlinear relationship between the measured physiological data and the CRI values, more sample waveforms may provide for a better comparison. From the comparison, a similarity coefficient may be calculated (e.g., using a least squares or similar analysis) to express the similarity between the sampled waveform and each of the reference waveforms. These similarity coefficients may be used to normalized and/or weight a CRI value corresponding to the respective waveform of reference data, and the CRI values as normalized/weighted by the similarity coefficients may be summed to produce an the determination of the presence of sepsis, severity of sepsis, and/or the effectiveness of treatments of sepsis.

In an aspect, normalizing the data can provide benefits in a clinical setting, because it can allow the clinician to quickly make a qualitative judgment of the patient's condition, while interpretation of the raw estimates/predictions might require additional analysis. Merely by way of example, with regard to the estimate of the compensatory reserve of the patient, that estimate might be normalized relative to a normative normal blood volume value corresponding to euvolemia and a normative minimum blood volume value corresponding to cardiovascular collapse. Once again, any values can be selected as the normative values. For example, if the normative normal blood volume is defined as 1, and the normative minimum blood volume value is defined as 0, the normalized value, falling between 0.0 and 1.0 can quickly apprise a clinician of the patient's location on a continuum between euvolemia and cardiovascular collapse. Similar normalizing procedures can be implemented for other estimated data (such as the determinations of sepsis, severity of sepsis, and the effectiveness of treatments, such as fluid resuscitation and/or vasopressors, and/or the like).

At block 430, the method 400 may further include displaying the data in real-time. As previously described with respect to the method 300, a display device may be configured to display, for example, values of SPS, V, P_{ƒ}, D_{VP}, P_{VP}, and/or P_{effVP} in real-time. In some embodiments, a normalized values of SPS, V, P_{ƒ}, D_{VP}, P_{VP}, and/or P_{effVP} may be displayed. Examples of values of SPS, V, P_{ƒ}, D_{VP}, P_{VP}, and/or P_{effVP}, may be as described above. For example, an SPS value of “0” may indicate the patient is not septic, whereas a an SPS value of 1 may indicate the patient is in septic shock. As with the display of CRI, the values of SPS, V, P_{ƒ}, D_{VP}, P_{VP}, and/or P_{effVP} may be displayed as and/or along with a “fuel gauge” type bar graph to quickly convey information via a color coding scheme.

At optional block 435, the method 400 may further include recommending a treatment based on the presence of sepsis, any of the parameters SPS, V, P_{ƒ}, D_{VP}, P_{VP}, and/or P_{effVP}, and/or the CRI waveform. The recommendation may similarly be displayed via the display device. The recommended treatment may include, without limitation, suggestions of a type of treatment (e.g., fluid resuscitation, vasopressors, antimicrobials), dosages (e.g., a volume of a bolus, a flow rate, etc.), changes to therapies or medications, etc.

At optional block 445, the method 400 may further include controlling a therapeutic device based on any of CRI, SPS, V, P_{ƒ}, D_{VP}, P_{VP}, and/or P_{effVP}, as described above with respect to the previous embodiments. In a specific, non-limiting, example, the method 400 might comprise controlling operation of an infusion pump, IV drip rate/flow rate, or other suitable therapeutic device based at least in part on the estimate of the patient's CRI. Merely by way of example, a computer system that performs the monitoring and estimating functions might also be configured to adjust a flow rate of an IV, and a volume of a bolus being administered to a patient based on the estimated CRI values of the patient. In other embodiments, the computer system might provide instructions or suggestions to a human operator of the IV pump, such as instructions to manually adjust a flow rate, etc.

In some embodiments, the method 400 might comprise repeating the operations of monitoring physiological data and/or CRI of the patient, and making determinations of sepsis and/or the effectiveness of a treatment of sepsis. Thus, displaying the data (and/or prediction), the patient's estimated CRI, specific determinations of sepsis, and/or effectiveness of treatment may be repeatedly estimated and/or predicted on any desired interval (e.g., after every heartbeat, every n number of seconds, etc.), on demand, before fluid resuscitation, during fluid resuscitation, after fluid resuscitation, before, during, and after the onset of sepsis, etc.

### Exemplary System and Hardware Implementation

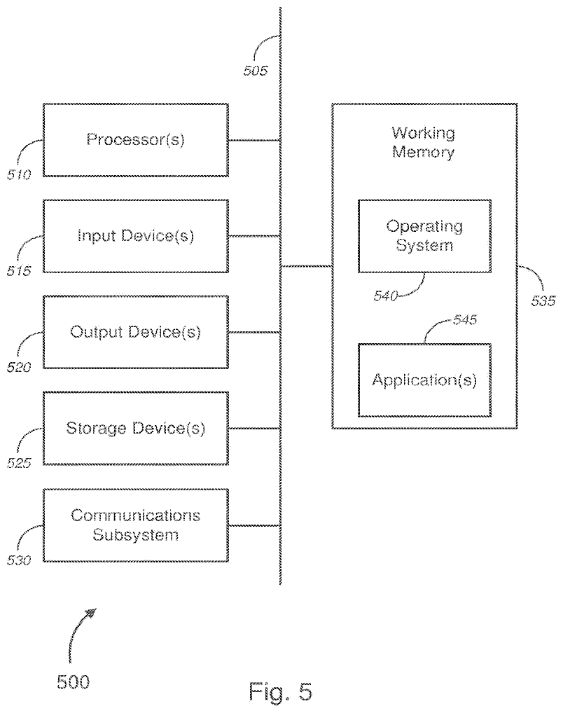

FIG. 5 is a block diagram illustrating an exemplary computer or system hardware architecture, in accordance with various embodiments. FIG. 5 provides a schematic illustration of one embodiment of a computer system 500 of the service provider system hardware that can perform the methods provided by various other embodiments, as described herein, and/or can perform the functions of computer or hardware system (i.e., sensor devices 105 and 310, user devices 120, computing system 125, computational device 205, monitoring computer 305, compensatory reserve index (“CRI”) server(s) 140, and therapeutic devices 215 and 315, etc.), as described above. It should be noted that FIG. 5 is meant only to provide a generalized illustration of various components, of which one or more (or none) of each may be utilized as appropriate. FIG. 5, therefore, broadly illustrates how individual system elements may be implemented in a relatively separated or relatively more integrated manner.

The computer or hardware system 500—which may represent an embodiment of the computer or hardware system (i.e., sensor devices 105 and 110, user devices 120, computing system 125, computational device 205, CRI server(s) 140, and therapeutic devices 215), described above with respect to FIGS. 1-4—is shown comprising hardware elements that can be electrically coupled via a bus 505 (or may otherwise be in communication, as appropriate). The hardware elements may include one or more processors 510, including, without limitation, one or more general-purpose processors and/or one or more special-purpose processors (such as microprocessors, digital signal processing chips, graphics acceleration processors, and/or the like); one or more input devices 515, which can include, without limitation, a mouse, a keyboard, and/or the like; and one or more output devices 520, which can include, without limitation, a display device, a printer, and/or the like.

The computer or hardware system 500 may further include (and/or be in communication with) one or more storage devices 525, which can comprise, without limitation, local and/or network accessible storage, and/or can include, without limitation, a disk drive, a drive array, an optical storage device, solid-state storage device such as a random access memory (“RAM”) and/or a read-only memory (“ROM”), which can be programmable, flash-updateable, and/or the like. Such storage devices may be configured to implement any appropriate data stores, including, without limitation, various file systems, database structures, and/or the like.

The computer or hardware system 500 may also include a communications subsystem 530, which can include, without limitation, a modem, a network card (wireless or wired), an infra-red communication device, a wireless communication device and/or chipset (such as a Bluetooth™ device, an 802.11 device, a WiFi device, a WiMax device, a WWAN device, cellular communication facilities, etc.), and/or the like. The communications subsystem 530 may permit data to be exchanged with a network (such as the network described below, to name one example), with other computer or hardware systems, and/or with any other devices described herein. In many embodiments, the computer or hardware system 500 will further comprise a working memory 535, which can include a RAM or ROM device, as described above.

The computer or hardware system 500 also may comprise software elements, shown as being currently located within the working memory 535, including an operating system 540, device drivers, executable libraries, and/or other code, such as one or more application programs 545, which may comprise computer programs provided by various embodiments (including, without limitation, hypervisors, VMs, and the like), and/or may be designed to implement methods, and/or configure systems, provided by other embodiments, as described herein. Merely by way of example, one or more procedures described with respect to the method(s) discussed above may be implemented as code and/or instructions executable by a computer (and/or a processor within a computer); in an aspect, then, such code and/or instructions can be used to configure and/or adapt a general purpose computer (or other device) to perform one or more operations in accordance with the described methods.

A set of these instructions and/or code may be encoded and/or stored on a non-transitory computer readable storage medium, such as the storage device(s) 525 described above. In some cases, the storage medium may be incorporated within a computer system, such as the system 500. In other embodiments, the storage medium may be separate from a computer system (i.e., a removable medium, such as a compact disc, etc.), and/or provided in an installation package, such that the storage medium can be used to program, configure, and/or adapt a general purpose computer with the instructions/code stored thereon. These instructions may take the form of executable code, which is executable by the computer or hardware system 500 and/or may take the form of source and/or installable code, which, upon compilation and/or installation on the computer or hardware system 500 (e.g., using any of a variety of generally available compilers, installation programs, compression/decompression utilities, etc.) then takes the form of executable code.

It will be apparent to those skilled in the art that substantial variations may be made in accordance with specific requirements. For example, customized hardware (such as programmable logic controllers, field-programmable gate arrays, application-specific integrated circuits, and/or the like) may also be used, and/or particular elements may be implemented in hardware, software (including portable software, such as applets, etc.), or both. Further, connection to other computing devices such as network input/output devices may be employed.

As mentioned above, in one aspect, some embodiments may employ a computer or hardware system (such as the computer or hardware system 500) to perform methods in accordance with various embodiments of the invention. According to a set of embodiments, some or all of the procedures of such methods are performed by the computer or hardware system 500 in response to processor 510 executing one or more sequences of one or more instructions (which may be incorporated into the operating system 540 and/or other code, such as an application program 545) contained in the working memory 535. Such instructions may be read into the working memory 535 from another computer readable medium, such as one or more of the storage device(s) 525. Merely by way of example, execution of the sequences of instructions contained in the working memory 535 may cause the processor(s) 510 to perform one or more procedures of the methods described herein.

The terms “machine readable medium” and “computer readable medium,” as used herein, refer to any medium that participates in providing data that causes a machine to operate in a specific fashion. In an embodiment implemented using the computer or hardware system 500, various computer readable media may be involved in providing instructions/code to processor(s) 510 for execution and/or may be used to store and/or carry such instructions/code (e.g., as signals). In many implementations, a computer readable medium is a non-transitory, physical, and/or tangible storage medium. In some embodiments, a computer readable medium may take many forms, including, but not limited to, non-volatile media, volatile media, or the like. Non-volatile media includes, for example, optical and/or magnetic disks, such as the storage device(s) 525. Volatile media includes, without limitation, dynamic memory, such as the working memory 535. In some alternative embodiments, a computer readable medium may take the form of transmission media, which includes, without limitation, coaxial cables, copper wire, and fiber optics, including the wires that comprise the bus 505, as well as the various components of the communication subsystem 530 (and/or the media by which the communications subsystem 530 provides communication with other devices). In an alternative set of embodiments, transmission media can also take the form of waves (including without limitation radio, acoustic, and/or light waves, such as those generated during radio-wave and infra-red data communications).

Common forms of physical and/or tangible computer readable media include, for example, a floppy disk, a flexible disk, a hard disk, magnetic tape, or any other magnetic medium, a CD-ROM, any other optical medium, punch cards, paper tape, any other physical medium with patterns of holes, a RAM, a PROM, and EPROM, a FLASH-EPROM, any other memory chip or cartridge, a carrier wave as described hereinafter, or any other medium from which a computer can read instructions and/or code.

Various forms of computer readable media may be involved in carrying one or more sequences of one or more instructions to the processor(s) 510 for execution. Merely by way of example, the instructions may initially be carried on a magnetic disk and/or optical disc of a remote computer. A remote computer may load the instructions into its dynamic memory and send the instructions as signals over a transmission medium to be received and/or executed by the computer or hardware system 500. These signals, which may be in the form of electromagnetic signals, acoustic signals, optical signals, and/or the like, are all examples of carrier waves on which instructions can be encoded, in accordance with various embodiments of the invention.

The communications subsystem 530 (and/or components thereof) generally will receive the signals, and the bus 505 then may carry the signals (and/or the data, instructions, etc. carried by the signals) to the working memory 535, from which the processor(s) 505 retrieves and executes the instructions. The instructions received by the working memory 535 may optionally be stored on a storage device 525 either before or after execution by the processor(s) 510.

While certain features and aspects have been described with respect to exemplary embodiments, one skilled in the art will recognize that numerous modifications are possible. For example, the methods and processes described herein may be implemented using hardware components, software components, and/or any combination thereof. Further, while various methods and processes described herein may be described with respect to particular structural and/or functional components for ease of description, methods provided by various embodiments are not limited to any particular structural and/or functional architecture but instead can be implemented on any suitable hardware, firmware and/or software configuration. Similarly, while certain functionality is ascribed to certain system components, unless the context dictates otherwise, this functionality can be distributed among various other system components in accordance with the several embodiments.

Moreover, while the procedures of the methods and processes described herein are described in a particular order for ease of description, unless the context dictates otherwise, various procedures may be reordered, added, and/or omitted in accordance with various embodiments. Moreover, the procedures described with respect to one method or process may be incorporated within other described methods or processes; likewise, system components described according to a particular structural architecture and/or with respect to one system may be organized in alternative structural architectures and/or incorporated within other described systems. Hence, while various embodiments are described with or without certain features for ease of description and to illustrate exemplary aspects of those embodiments, the various components and/or features described herein with respect to a particular embodiment can be substituted, added and/or subtracted from among other described embodiments, unless the context dictates otherwise. Consequently, although several exemplary embodiments are described above, it will be appreciated that the invention is intended to cover all modifications and equivalents within the scope of the following claims.

What is claimed is:

1. A method for determining whether a patient is septic, the method comprising:
obtaining, with one or more sensors disposed in a sensor device, physiological data of a patient continuously over a first time period, the physiological data comprising non-invasively obtained waveforms of physiological data;
determining, via a computer system, based on the physiological data, a hemodynamic parameter of the patient over the first time period, wherein the hemodynamic parameter is a patient-specific indication of the patient's proximity to hemodynamic decompensation at a given time, wherein the hemodynamic parameter is a numerical value indicating a relationship between an intravascular volume loss of a patient at the given time and an intravascular volume loss at hemodynamic decompensation of the patient,
wherein determining the hemodynamic parameter of the patient comprises:
applying a hemodynamic model to the physiological data, the hemodynamic model relating the physiological data to the hemodynamic parameter, wherein the hemodynamic model comprises a plurality waveforms of reference data;
comparing one or more waveforms of the physiological data of the patient to the each of the plurality of waveforms of reference data, each of the plurality of waveforms of reference data corresponding to a respective value of the hemodynamic parameter;
determining the hemodynamic parameter of the patient based on the comparison to each of the plurality of waveforms of reference data;
determining, via the computer system, based on the hemodynamic parameter of the patient over the first time period, whether the patient is septic, wherein determining whether the patient is septic further comprises:
applying a sepsis model to the hemodynamic parameter of the patient over the first time period, wherein the hemodynamic parameter over the first time period is a waveform of the hemodynamic parameter of the patient, the sepsis model relating waveforms of the hemodynamic parameter to a sepsis value representing whether the patient is septic, wherein sepsis model comprises a plurality of reference waveforms of the hemodynamic parameter;
comparing the waveform of the hemodynamic parameter over the first time period to each of the plurality of reference waveforms of the hemodynamic parameter, each of the plurality of reference waveforms of the hemodynamic parameter corresponding to a respective sepsis value; and
determining whether the patient is septic based on the sepsis value of the patient; and
displaying, on a display screen of a user device, at least one of the hemodynamic parameter of the patient and a determination of whether the patient is septic.

2. The method of claim 1, further comprising:
determining, based on the hemodynamic parameter of the patient over the first time period, whether sepsis in the patient is fluid responsive, wherein determining whether sepsis in the patient is fluid responsive further comprises:
applying a fluid responsivity model to the hemodynamic parameter of the patient over the first time period, wherein the hemodynamic parameter over the first time period is a waveform of the hemodynamic parameter of the patient, the fluid responsivity model relating waveforms of the hemodynamic parameter to a fluid responsivity value indicating at least one of a volume of fluid that should be administered to the patient or a probability that the patient requires additional fluid to be administered, wherein the fluid responsivity model comprises a plurality of reference waveforms of the hemodynamic parameter;
comparing the waveform of the hemodynamic parameter of the patient over the first time period to each of the plurality of reference waveforms of the hemodynamic parameter, each of the plurality of reference waveforms of the hemodynamic parameter corresponding to the fluid responsivity value; and
determining whether the sepsis is fluid responsive based on the fluid responsivity value of the patient.

3. The method of claim 1, further comprising:
determining, based on the hemodynamic parameter of the patient over the first time period, whether a sepsis treatment is effective, wherein the sepsis treatment includes administering a therapeutic agent, wherein determining whether the sepsis treatment is effective further comprises:
applying a treatment effectiveness model to the hemodynamic parameter of the patient over the first time period, wherein the hemodynamic parameter over the first time period is a waveform of hemodynamic parameter of the patient, the treatment effectiveness model relating waveforms of the hemodynamic parameter to a treatment effectiveness value indicating at least one of a dosage of the therapeutic agent or a probability that additional therapeutic agent should be administered, wherein the treatment effectiveness model comprises a plurality of reference waveforms of the hemodynamic parameter;
comparing the waveform of the hemodynamic parameter of the patient over the first time period to each of the plurality of reference waveforms of the hemodynamic parameter, each of the plurality of reference waveforms of the hemodynamic parameter corresponding to the treatment effectiveness value; and
determining whether the sepsis treatment is effective based on the treatment effectiveness value of the patient.

4. The method of claim 3, further comprising:
controlling a therapeutic device based on the treatment effectiveness value of the patient, wherein the controlling the therapeutic device includes adjusting at least one of a dosage of the therapeutic agent or a volume of fluid administered to the patient.

5. The method of claim 1, wherein determining a sepsis value of the patient further comprises determining the sepsis value based on changes in the hemodynamic parameter of the patient over the first time period, wherein the changes in the hemodynamic parameter in the first time period are compared to changes in reference waveforms of the hemodynamic parameter.

6. The method of claim 1, wherein determining a sepsis value further comprises:
producing, for the waveform of the hemodynamic parameter of the patient, a respective similarity coefficient expressing a similarity between the waveform of the hemodynamic parameter of the patient over the first time period and each of the plurality of reference waveforms of the hemodynamic parameter of the sepsis model;
normalizing, with the computer system, the respective similarity coefficients for each of the plurality of reference waveforms of the hemodynamic parameter of the sepsis model; and
summing, each respective sepsis value, corresponding to each of the plurality of reference waveforms of the hemodynamic parameter, respectively weighted by the normalized similarity coefficient for each of the reference waveforms of the hemodynamic parameter; and
determining the sepsis value of the patient based on the sum of the respectively weighted sepsis values as weighted by the normalized similarity coefficients.

7. The method of claim 1, further comprising:
determining a stage of sepsis of the patient based on the sepsis value, wherein the sepsis value corresponds to a stage of sepsis of the patient, wherein a range of sepsis values corresponds to respective stages of sepsis, wherein the stages of sepsis correspond to a severity of the sepsis from no sepsis being the least severe, to septic shock being most severe.

8. The method of claim 1, further comprising:
differentiating sepsis in the patient from other forms of infection based on the sepsis value, wherein the sepsis value further differentiates sepsis from other forms of infection, wherein the sepsis value corresponding to no sepsis may further indicate that a different form of infection is present.

9. The method of claim 1, wherein the hemodynamic parameter is an estimated compensatory reserve index (CRI) value of the patient over the first time period, wherein CRI is a value expressing a relationship given by the following formula:
$$CRI ( t ) = 1 \frac{B L V ( t )}{B L {V}_{HDD}}$$
wherein CRI(t) is the compensatory reserve at time t, BLV(t) is an intravascular volume loss at time t, and BLV_{HDD} is an intravascular volume loss at a point of hemodynamic decompensation.

10. An apparatus, comprising:
a processor; and
a non-transitory computer readable medium in communication with the processor, the non-transitory computer readable medium having encoded thereon a set of instructions executable by the processor to:
obtain, with one or more sensors disposed in a sensor device, physiological data of a user continuously over a first time period, the physiological data comprising non-invasively obtained waveforms of physiological data;
determine based on the physiological data, a hemodynamic parameter of the patient over the first time period, wherein the hemodynamic parameter is a patient-specific indication of the patient's proximity to hemodynamic decompensation at a given time, wherein the hemodynamic parameter is a numerical value indicating a relationship between an intravascular volume loss of a patient at the given time and an intravascular volume loss at hemodynamic decompensation of the patient, wherein determining the hemodynamic parameter of the patient comprises:
applying a hemodynamic model to the physiological data, the hemodynamic model relating the physiological data to the hemodynamic parameter, wherein the hemodynamic model comprises a plurality waveforms of reference data;
comparing one or more waveforms of the physiological data of the patient to the each of the plurality of waveforms of reference data, each of the plurality of waveforms of reference data corresponding to a respective value of the hemodynamic parameter;
determining the hemodynamic parameter of the patient based on the comparison to each of the plurality of waveforms of reference data;
determine based on the hemodynamic parameter of the patient over the first time period, whether the patient is septic, wherein determining whether the patient is septic further comprises:
applying a sepsis model to the hemodynamic parameter of the patient over the first time period, wherein the hemodynamic parameter over the first time period is a waveform of the hemodynamic parameter of the patient, the sepsis model relating waveforms of the hemodynamic parameter to a sepsis value representing whether the patient is septic, wherein sepsis model comprises a plurality of reference waveforms of the hemodynamic parameter;
comparing the waveform of the hemodynamic parameter of the patient over the first time period to each of the plurality of reference waveforms of the hemodynamic parameter, each of the plurality of reference waveforms of the hemodynamic parameter corresponding to a respective sepsis value; and
determining whether the patient is septic based on the sepsis value of the patient; and
display, on a display screen of a user device, at least one of the hemodynamic parameter of the patient and a determination of whether the patient is septic.

11. The apparatus of claim 10, wherein the set of instructions is further executable by the processor to:
determine, based on the hemodynamic parameter of the patient over the first time period, whether sepsis in the patient is fluid responsive, wherein determining whether sepsis in the patient is fluid responsive further comprises:
applying a fluid responsivity model to the hemodynamic parameter of the patient over the first time period, wherein the hemodynamic parameter over the first time period is a waveform of the hemodynamic parameter of the patient, the fluid responsivity model relating waveforms of the hemodynamic parameter to a fluid responsivity value indicating at least one of a volume of fluid that should be administered to the patient or a probability that the patient requires additional fluid to be administered, wherein the fluid responsivity model comprises a plurality of reference waveforms of the hemodynamic parameter;
comparing the waveform of the hemodynamic parameter of the patient over the first time period to each of the plurality of reference waveforms of the hemodynamic parameter, each of the plurality of reference waveforms of the hemodynamic parameter corresponding to the fluid responsivity value; and
determining whether the sepsis is fluid responsive based on the fluid responsivity value of the patient.

12. The apparatus of claim 10, wherein the set of instructions is further executable by the processor to:
determine, based on the hemodynamic parameter of the patient over the first time period, whether a sepsis treatment is effective, wherein the sepsis treatment includes administering a therapeutic agent, wherein determining whether the sepsis treatment is effective further comprises:
applying a treatment effectiveness model to the hemodynamic parameter of the patient over the first time period, wherein the hemodynamic parameter over the first time period is a waveform of the hemodynamic parameter of the patient, the treatment effectiveness model relating waveforms of the hemodynamic parameter to a treatment effectiveness value indicating at least one of a dosage of the therapeutic agent or a probability that additional therapeutic agent should be administered, wherein the treatment effectiveness model comprises a plurality of reference waveforms of the hemodynamic parameter;
comparing the waveform of the hemodynamic parameter of the patient over the first time period to each of the plurality of reference waveforms of the hemodynamic parameter, each of the plurality of reference waveforms of the hemodynamic parameter corresponding to the treatment effectiveness value; and
determining whether the sepsis treatment is effective based on the treatment effectiveness value of the patient.

13. The apparatus of claim 12, wherein the therapeutic agent includes a vasopressor, wherein the set of instructions is further executable by the processor to:
control a therapeutic device based on the treatment effectiveness value of the patient, wherein the controlling the therapeutic device includes adjusting at least one of a dosage of the therapeutic agent or a volume of fluid administered to the patient.

14. The apparatus of claim 10, wherein determining a sepsis value further comprises instructions executable by the processor to:
produce, for the waveform of the hemodynamic parameter of the patient, a respective similarity coefficient expressing a similarity between the waveform of the hemodynamic parameter of the patient over the first time period and each of the plurality of reference waveforms of the hemodynamic parameter of the sepsis model;
normalize, with the computer system, the respective similarity coefficients for each of the plurality of reference waveforms of the hemodynamic parameter of the sepsis model; and
sum, each respective sepsis value, corresponding to each of the plurality of reference waveforms of the hemodynamic parameter, respectively weighted by the normalized similarity coefficient for each of the reference waveforms of the hemodynamic parameter; and
determine the sepsis value of the patient based on the sum of the respectively weighted sepsis values as weighted by the normalized similarity coefficients.

15. The apparatus of claim 10, wherein the set of instructions is further executable by the processor to:
determine a stage of sepsis of the patient based on the sepsis value, wherein the sepsis value corresponds to a stage of sepsis of the patient, wherein a range of sepsis values corresponds to respective stages of sepsis, wherein the stages of sepsis correspond to a severity of the sepsis from no sepsis being the least severe, to septic shock being most severe.

16. The apparatus of claim 10, wherein the hemodynamic parameter is an estimated compensatory reserve index (CRI) value of the patient over the first time period, wherein CRI is a value expressing a relationship given by the following formula:
$$CRI ( t ) = 1 \frac{B L V ( t )}{B L {V}_{HDD}}$$
wherein CRI(t) is the compensatory reserve at time t, BLV(t) is an intravascular volume loss at time t, and BLV_{HDD} is an intravascular volume loss at a point of hemodynamic decompensation.

17. A system, comprising:
one or more sensors configured to obtain physiological data from a patient, the physiological data comprising non-invasively obtained waveforms of physiological data;
a computer system in communication with the one or more sensors, the computer system comprising:
a processor; and
a non-transitory computer readable medium in communication with the processor, the non-transitory computer readable medium having encoded thereon a set of instructions executable by the processor to:
obtain, with one or more sensors disposed in a sensor device, physiological data of a user continuously over a first time period, the physiological data comprising non-invasively obtained waveforms of physiological data;
a hemodynamic parameter of the patient over the first time period, wherein the hemodynamic parameter is a patient-specific indication of the patient's proximity to hemodynamic decompensation at a given time, wherein the hemodynamic parameter is a numerical value indicating a relationship between an intravascular volume loss of a patient at the given time and an intravascular volume loss at hemodynamic decompensation of the patient, wherein determining the hemodynamic parameter of the patient comprises:
applying a hemodynamic model to the physiological data, the hemodynamic model relating the physiological data to a value of the hemodynamic parameter, wherein the hemodynamic model comprises a plurality waveforms of reference data;
comparing one or more waveforms of the physiological data of the patient to the each of the plurality of waveforms of reference data, each of the plurality of waveforms of reference data corresponding to a respective value of the hemodynamic parameter;
determining the hemodynamic parameter of the patient based on the comparison to each of the plurality of waveforms of reference data;
determine based on the hemodynamic parameter of the patient over the first time period, whether the patient is septic, wherein determining whether the patient is septic further comprises:
applying a sepsis model to the hemodynamic parameter of the patient over the first time period, wherein the hemodynamic parameter over the first time period is a waveform of the hemodynamic parameter of the patient, the sepsis model relating waveforms of the hemodynamic parameter to a sepsis value representing whether the patient is septic, wherein sepsis model comprises a plurality of reference waveforms of the hemodynamic parameter;
comparing the waveform of the hemodynamic parameter of the patient over the first time period to each of the plurality of reference waveforms of the hemodynamic parameter, each of the plurality of reference waveforms of the hemodynamic parameter corresponding to a respective sepsis value; and
determining whether the patient is septic based on the sepsis value of the patient; and
display, on a display screen of a user device, at least one of the hemodynamic parameter of the patient and a determination of whether the patient is septic.

18. The system of claim 17, wherein the set of instructions is further executable by the processor to:
determine, based on the hemodynamic parameter of the patient over the first time period, whether sepsis in the patient is fluid responsive, wherein determining whether sepsis in the patient is fluid responsive further comprises:
applying a fluid responsivity model to the hemodynamic parameter of the patient over the first time period, wherein the hemodynamic parameter over the first time period is a waveform of the hemodynamic parameter of the patient, the fluid responsivity model relating waveforms of the hemodynamic parameter to a fluid responsivity value indicating at least one of a volume of fluid that should be administered to the patient or a probability that the patient requires additional fluid to be administered, wherein the fluid responsivity model comprises a plurality of reference waveforms of the hemodynamic parameter;
comparing the waveform of the hemodynamic parameter of the patient over the first time period to each of the plurality of reference waveforms of the hemodynamic parameter, each of the plurality of reference waveforms of the hemodynamic parameter corresponding to the fluid responsivity value; and
determining whether the sepsis is fluid responsive based on the fluid responsivity value of the patient.

19. The system of claim 17, wherein the set of instructions is further executable by the processor to:
determine, based on the hemodynamic parameter of the patient over the first time period, whether a sepsis treatment is effective, wherein the sepsis treatment includes administering a therapeutic agent, wherein determining whether the sepsis treatment is effective further comprises:
applying a treatment effectiveness model to the hemodynamic parameter of the patient over the first time period, wherein the hemodynamic parameter over the first time period is a waveform of the hemodynamic parameter of the patient, the treatment effectiveness model relating waveforms of the hemodynamic parameter to a treatment effectiveness value indicating at least one of a dosage of the therapeutic agent or a probability that additional therapeutic agent should be administered, wherein the treatment effectiveness model comprises a plurality of reference waveforms of the hemodynamic parameter;
comparing the waveform of the hemodynamic parameter of the patient over the first time period to each of the plurality of reference waveforms of the hemodynamic parameter, each of the plurality of reference waveforms of the hemodynamic parameter corresponding to the treatment effectiveness value; and
determining whether the sepsis treatment is effective based on the treatment effectiveness value of the patient.

20. The system of claim 17, wherein the therapeutic agent includes a vasopressor, wherein the set of instructions is further executable by the processor to:
control a therapeutic device based on the treatment effectiveness value of the patient, wherein the controlling the therapeutic device includes adjusting at least one of a dosage of the therapeutic agent or a volume of fluid administered to the patient.

21. The system of claim 17, wherein the set of instructions is further executable by the processor to:
determine a stage of sepsis of the patient based on the sepsis value, wherein the sepsis value corresponds to a stage of sepsis of the patient, wherein a range of sepsis values corresponds to respective stages of sepsis, wherein the stages of sepsis correspond to a severity of the sepsis from no sepsis being the least severe, to septic shock being most severe.

22. The system of claim 17, wherein the hemodynamic parameter is an estimated compensatory reserve index (CRI) value of the patient over the first time period, wherein CRI is a value expressing a relationship given by the following formula:
$$CRI ( t ) = 1 \frac{B L V ( t )}{B L {V}_{HDD}}$$
wherein CRI(t) is the compensatory reserve at time t, BLV(t) is an intravascular volume loss at time t, and BLV_{HDD} is an intravascular volume loss at a point of hemodynamic decompensation.
